(19) United States
(12) Patent Application Publication
(10) Pub. No.: US 2006/0116518 A1
(43) Pub. Date: Jun. 1, 2006

(54) Novel phenanthridines

(75) Inventors: Dieter Flockerzi, Allensbach (DE); Beate Schmidt, Allensbacher (DE); Steffen Weinbrenner, Konstanz (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 10/524,634

(22) PCT Filed: Aug. 13, 2003
(86) PCT No.: PCT/EP03/08967

§ 371 Date: Feb. 16, 2005

(30) Foreign Application Priority Data
Aug. 17, 2002 (EP) 02018530.2

(51) Int. Cl. C07D 221/12; A61K 31/473

(52) U.S. Cl. 546/108; 514/298

(57) ABSTRACT

Compounds of a certain formula 1, 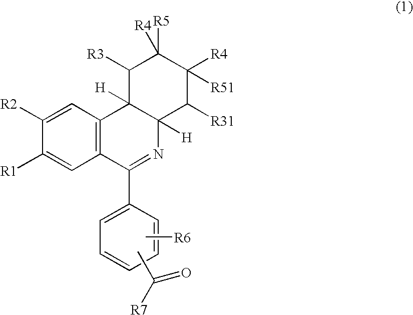 in which R1, R2, R3, R31, R4, R5, R51, R6 and R7 have the meanings indicated in the description, are novel effective PDE4 inhibitors.

### FIELD OF APPLICATION OF THE INVENTION

The invention relates to novel 6-phenylphenanthridines, which are used in the pharmaceutical industry for the production of pharmaceutical compositions.

### KNOWN TECHNICAL BACKGROUND

The International Patent applications WO97/28131 (=U.S. Pat. No. 6,191,138), WO97/35854 (=U.S. Pat. No. 6,127,378), WO99/05113 (=U.S. Pat. No. 6,121,279), WO99/05111 (=U.S. Pat. No. 6,410,551), WO00/42018, WO00/42019, WO00/42020, WO02/05616, WO02/06238 and WO02/06270 describe 6-phenylphenanthridines as PDE4 inhibitors. In the International Patent application WO02/066476 benzonaphthyridine derivatives are described which have a guanidyl substituent In the International Patent application WO01/70746 furoisoquinoline derivatives are described as PDE4 inhibitors. In the European Patent application EP 0490823 dihydroisoquinoline derivatives are described which are useful in the treatment of asthma.

### DESCRIPTION OF THE INVENTION

It has now been found that the compounds of formula 1, which are described in more detail below and which differ from the prior-art compounds in particular in the substitution pattern on the 6-phenyl ring, have surprising and particularly advantageous properties.

The invention thus relates to compounds of formula 1, 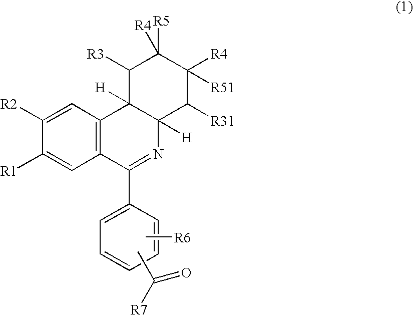
in which
R1 is hydroxyl, 1-4C-alkoxy, 3-7C-cycloalkoxy, 3-7C-cycloalkylmethoxy or completely or predominantly fluorine-substituted 1-4C-alkoxy, and
R2 is hydroxyl, 1-4C-alkoxy, 3-7C-cycloalkoxy, 3-7C-cycloalkylmethoxy or completely or predominantly fluorine-substituted 1-4C-alkoxy,
R1 and R2 together are a 1-2C-alkylenedioxy group,
R3 is hydrogen or 1-4C-alkyl,
R31 is hydrogen or 1-4C-alkyl,
or in which
R3 and R31 together are a 1-4C-alkylene group,
R4 is hydrogen or 1-4C-alkyl,
R5 is hydrogen,
R51 is hydrogen,
or in which
R5 and R51 together represent an additional bond,
R6 is hydrogen, halogen, nitro, 1-4C-alkyl, trifluoromethyl or 1-4C-alkoxy,
R7 is a radical of formulae (a), (b), (c) or (d) 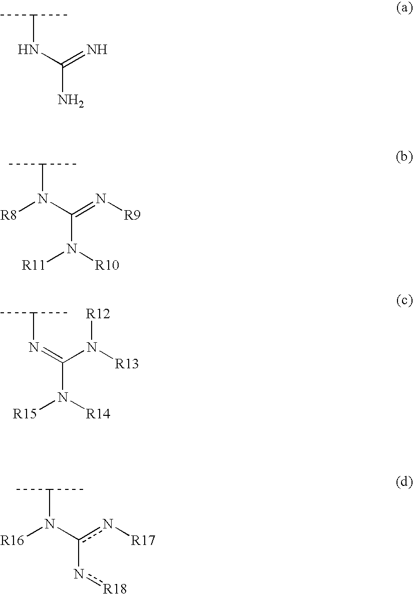
   in which
if R7 is a radical of the formula (b),
either
   R8, R9, R10 and R11 independently of one another are hydrogen, 1-7C-alkyl, 3-7C-cycloalkyl, 3-7C-cycloalkylmethyl, cyano, hydroxy-2-4C-alkyl, 1-4C-alkoxy-2-4C-alkyl or R28,
or
   R8 is hydrogen, 1-7C-alkyl, 3-7C-cycloalkyl, 3-7C-cycloalkylmethyl, hydroxy-2-4C-alkyl, 1-4C-alkoxy-2-4C-alkyl or R28,
   R9 is is hydrogen, 1-7C-alkyl, 3-7C-cycloalkyl, 3-7C-cycloalkylmethyl, hydroxy-2-4C-alkyl, 1-4C-alkoxy-2-4C-alkyl or R28, and
   R10 and R11, together and including the nitrogen atom to which both are bonded, are a pyrrolidin-1-yl, piperidin-1-yl, azepan-1-yl, azocan-1-yl, azonan-1-yl, azecan-1-yl, morpholin-4-yl, tetrahydroisoquinolin-2-yl, tetrahydro6,7-dimethoxyisoquinolin-2-yl, 3,5dimethyl-pyrazol1-yl, pyrazol-1-yl, 2,6-dimethyl-morpholin-4-yl, 2,6-dimethyl-piperidin-1-yl, 4-benzyl-piperidin-1-yl, thiomorpholin-4-yl or 1H-1,2,4-triazol-1-yl radical, or a piperazin-1-yl radical substituted in 4-position by R19,
or
   R8 is hydrogen, 1-7C-alkyl, 3-7C-cycloalkyl, 3-7C-cycloalkylmethyl, hydroxy-2-4C-alkyl, 1-4C-alkoxy-2-4C-alkyl or R28,
   R9 is hydrogen, 1-7C-alkyl, 3-7C-cycloalkyl, 3-7C-cycloalkylmethyl, hydroxy-2-4C-alkyl, 1-4C-alkoxy-2-4C-alkyl or R28,
   R10 is hydrogen, 1-7C-alkyl, 3-7C-cycloalkyl, 3-7C-cycloalkylmethyl, hydroxy-2-4C-alkyl, 1-4C-alkoxy-2-4C-alkyl or R28, and
   R11 is Aryl1, naphthyl, phenyl, phenyl substituted by R20 and/or R21, pheny -1-4C-alkyl or phenyl-1-4C-alkyl substituted by R22 and R23,
      in which
if R7 is a radical of the formula (c),
either
   R12, R13, R14 and R15 independently of one another are hydrogen, 1-7C-alkyl, 3-7C-cycloalkyl, 3-7C-cycloalkylmethyl, hydroxy-2-4C-alkyl,1-4C-alkoxy-24C-alkyl or R28,
      or
   R12 and R13 independently of one another are hydrogen, 1-7C-alkyl, 3-7C-cycloalkyl, 3-7C-cycloalkylmethyl, hydroxy-2-4C-alkyl, 1-4C-alkoxy-2-4C-alkyl or R28, and
   R14 and R15, together and including the nitrogen atom to which both are bonded, are a pyrrolidin-1-yl, piperidin-1-yl, azepan-1-yl, azocan-1-yl, azonan-1-yl, azecan-1-yl, morpholin-4-yl, tetrahydroisoquinolin-2-yl, tetrahydro,6,7-dimethoxyisoquinolin-2-yl, 3,5-dimethyl-pyrazol-1-yl, pyrazol-1-yl, 2,6-dimethyl-morpholin-4-yl, 2,6-dimethyl-piperidin-1-yl, 4-benzyl-piperidin-1-yl, thiomorpholin-4-yl or 1H-1,2,4-triazol-1-yl radical, or a piperazin-1-yl radical substituted in 4-position by R19,
      or
   R12 and R13, together and including the nitrogen atom to which both are bonded, are a pyrrolidin-1-yl, piperidin-1-yl, azepan-1-yl, morpholino-4-yl, 4-(1-4C-alkyl-)-piperazin-1-yl, 2,6-dimethylmorpholin-4-yl, 2,6-dimethyl-piperidin-1-yl, 4-benzyl-piperidin-1-yl or thiomorpholin-4-yl radical, and
   R14 and R15, together and including the nitrogen atom to which both are bonded, are a pyrrolidin-1-yl, piperidin-1-yl, azepan-1-yl, morpholino-4-yl, 4-(1-4C-alkyl-)-piperazin-1-yl, 2,6-dimethyl-morpholin-4-yl, 2,6-dimethyl-piperidin-1-yl, 4-benzyl-piperidin-1-yl or thiomorpholin-4-yl radical,
      or
   R12 and R15 independently of one another are hydrogen or 1-4C-alkyl, and
   R13 and R14, together and with inclusion of the N—C(═)—N structure to which they are bonded, are a hexahydropyrimidin-2-ylidene or imidazolidin-2-ylidene radical,
      in which
if R7 is a radical of the formula (d),
   R16 is hydrogen, 1-7C-alkyl, 3-7C-cycloalkyl, 3-7C-cycloalkylmethyl, hydroxy-2-4C-alkyl, 1-4C-alkoxy-2-4C-alkyl or R28, and
   R17 and R18, together and with inclusion of the N—C(—)—N structure to which they are bonded are Aryl2,
Aryl1 is 4-methylthiazol-2-yl, benzimidazol-2-yl, 5-nitrobenzimidazol-2-yl, 5-chlorobenzimidazol-2-yl, 5-methylbenzimidazol-2-yl, 4-methylquinazolin-2-yl, benzothiazol-2-yl, benzoxazol-2-yl or pyrimidin-2-yl,
Aryl2 is 1-methyl-4-oxo-4,5-dihydro-1H-imidazol-2-yl, imidazol-2-yl, 4,5-dicyano-imidazol-2-yl, 4-methyl-imidazol-2-yl, 4-ethyl-benzimidazol-2-yl, 4-acetyl-imidazol-2-yl, 1H-[1,2,4]triazol-3-yl, benzimidazol-2-yl, 1-methyl-benzimidazol-2-yl, 1-ethyl-benzimidazol-2-yl, 5,6-dimethyl-benzimidazol-2-yl, purin-8-yl, 6-amino-7-methyl-7H-purine-8yl, 1,6-dimethylimidazo[4,5-b]pyridin-2-yl, 1,5,6-trimethylimidazo[4,5-b]pyridin-2-yl, 1,3-dimethyl-3,7-dihydro-1H-purine-2,6-dione-8-yl, 7-ethyl-3-methyl-3,7-dihydro-purine-2,6-dione-8-yl, 1,3,7-trimethyl-3,7-dihydro-purine-2,6-dione-8-yl, thiadiazolyl, 1,4-dihydrotetrazol-5-yl, 2H-[1,2,4]triazol-3-yl, 1,3-dihydrobenzimidazol-5-yl, 1H-tetrazol-5-yl, pyrimidin-2-yl or 4,6-dimethyl-pyrimidin-2-yl,
R19 is 1-4C-alkyl, formyl, 3-7C-cycloalkyl, 3-7C-cycloalkylmethyl, 1-4C-alkoxycarbonyl-1-4C-alkyl, 1-4C-alkylcarbonyl, hydroxy-2-4C-alkyl, 1-4C-alkoxy-2-4C-alkyl, hydroxy-2-4C-alkoxy-2-4C-alkyl, 1-4C-alkoxy-2-4C-alkoxy-2-4C-alkyl, phenyl, phenyl substituted by R24 and/or R25, [benzo(1,3)dioxol]-5-ylmethyl, phenyl-1-4C-alkyl or phenyl-1-4C-alkyl substituted in the phenyl moiety by R26 and/or R27,
R20 is halogen, nitro, carboxyl, 1-4C-alkyl, trifluoromethyl or 1-4C-alkoxy,
R21 is halogen, 1-4C-alkyl or 1-4C-alkoxy,
R22 is halogen, nitro, carboxyl, 1-4C-alkyl, trifluoromethyl or 1-4C-alkoxy,
R23 is halogen, 1-4C-alkyl or 1-4C-alkoxy,
R24 is halogen, nitro, carboxyl, 1-4C-alkyl, 1-4C-alkylcarbonyl, trifluoromethyl or 1-4C-alkoxy,
R25 is halogen, 1-4C-alkyl or 1-4C-alkoxy,
R26 is halogen, nitro, carboxyl, 1-4C-alkyl, trifluoromethyl or 1-4C-alkoxy,
R27 is halogen, 1-4C-alkyl or 1-4C-alkoxy,
R28 is R29(R30)N-2-4C-alkyl wherein
R29 and R30, together and including the nitrogen atom to which both are bonded, are a pyrrolidin-1-yl, piperidin-1-yl, piperazin-1-yl, 4-(1-4C-alkyl-)piperazin-1-yl, azepan-1yl, azocan-1-yl, azonan-1-yl, azecan-1-yl, tetrahydroisoquinolin-2-yl, tetrahydro-6,7-dimethoxyisoquinolin-2-yl, 3,5-dimethyl-pyrazol-1-yl, pyrazol-1-yl, morpholin-4-yl, 2,6-dimethyl-morpholin-4-yl, 2,6-dimethyl-piperidin-1-yl, 4-benzyl-piperidin-1-yl, thiomorpholin-4-yl or 1H-1,2,4-triazol-1-yl radical,
the salts of these compounds, as well as the N-oxides, enantiomers, E/Z isomers and tautomers of these compounds and their salts.

1-4C-Alkyl represents a straight-chain or branched alkyl radical having 1 to 4 carbon atoms. Examples which may be mentioned are the butyl, isobutyl, sec-butyl, tert-butyl, propyl, isopropyl and, preferably, the ethyl and methyl radicals.

2-4C-Alkyl represents a straight-chain or branched alkyl radical having 2 to 4 carbon atoms. Examples which may be mentioned are the butyl, isobutyl, sec-butyl, tert-butyl, propyl, isopropyl and, preferably, the ethyl radicals.

1-4C-Alkoxy represents radicals which, in addition to the oxygen atom, contain a straight-chain or branched alkyl radical having 1 to 4 carbon atoms. Examples which may be mentioned are the butoxy, iso-butoxy, sec-butoxy, tert-butoxy, propoxy, isopropoxy and, preferably, the ethoxy and methoxy radicals.

3-7C-Cycloalkoxy represents, for example, cyclopropyloxy, cyclobutyloxy, cyclopentyloxy, cyclohexyloxy and cycloheptyloxy, of which cyclopropyloxy, cyclobutyloxy and cyclopentyloxy are preferred.

3-7C-Cycloalkylmethoxy represents, for example, cyclopropylmethoxy, cyclobutylmethoxy, cyclopentylmethoxy, cyclohexylmethoxy and cycloheptylmethoxy, of which cyclopropylmethoxy, cyclobutylmethoxy and cyclopentylmethoxy are preferred.

As 1-4C-Alkoxy which is completely or predominantly substituted by fluorine, the 2,2,3,3,3-pentafluoropropoxy, the perfluoroethoxy, the 1,1,2,2-tetrafluoroethoxy, the 1,2,2-trifluoroethoxy, the trifluoromethoxy, in particular the 2,2,2-trifluoroethoxy, and preferably the difluoromethoxy radicals, for example, may be mentioned. In this context, “predominantly” means that more than half of the hydrogen atoms of the 1-4C-alkoxy groups are replaced by fluorine atoms.

1-2C-Alkylenedioxy represents, for example, the methylenedioxy (—O—CH₂—O—) or the ethylenedioxy (—O—CH₂-CH₂—O—) radical.

If R3 and R31 together have the meaning 1-4C-alkylene, the positions 1 and 4 in compounds of the formula 1 are linked to one another by a 1-4C-alkylene bridge, 1-4C-alkylene representing straight-chain or branched alkylene radicals having 1 to 4 carbon atoms. Examples which may be mentioned are the radicals methylene [—CH₂—], ethylene [—CH₂—CH₂—], trimethylene [—CH₂—CH₂CH_{2—], 1,2}-dimethylethylene [—CH(CH₃)—CH(CH₃)—] and isopropylidene [—C(CH₃)₂—].

Halogen within the meaning of the invention is fluorine, chlorine or bromine.

1-7C-Alkyl represents straight-chain or branched alkyl radicals having 1 to 7 carbon atoms. Examples which may be mentioned are the heptyl, isoheptyl (5methylhexyl), hexyl, isohexyl (4-methylpentyl), neohexyl (3,3-dimethylbutyl), pentyl, isopentyl (3-methylbutyl), neopentyl (2,2-dimethylpropyl), butyl, isobutyl, sec-butyl, tert-butyl, propyl, isopropyl, ethyl or methyl radical.

3-7C-Cycloalkyl represents the cyclopropyl, cyclobutyl, cyclopentyl, cyclohexyl or cycloheptyl radical. 3-7C-Cycloalkylmethyl represents a methyl radical which is substituted by one of the abovementioned 3-7Cycloalkyl radicals. Examples which may be mentioned are the cycloalkylmethyl radicals cyclopropylmethyl, cyclobutylmethyl and cyclopentylmethyl.

Hydroxy-2-4C-alkyl represents 2-4C-alkyl radicals which are substituted by a hydroxyl group. Examples which may be mentioned are the 2-hydroxyethyl and the 3-hydroxypropyl radicals.

An example which may be mentioned for a hydroxy-2-4C-alkoxy-2-4C-alkyl radical is the (2-hydroxyethoxy)ethyl radical.

An example of a 1-4C-alkoxy-2-4C-alkoxy-2-4C-alkyl radical is the (2-methoxyethoxy)ethyl radical.

1-4C-Alkylcarbonyl is a carbonyl group to which one of the abovementioned 1-4C-alkyl radicals is bonded. An example is the acetyl radical [CH₃C(O)-].

1-4C-Alkoxycarbonyl is a carbonyl group to which one of the abovementioned 1-4C-alkoxy radicals is bonded. Examples are the methoxycarbonyl [CH₃O—C(O)—] and the ethoxycarbonyl [CH₃CH₂O—C(O)—] radical.

1-4C-Alkoxycarbonyl-1-4C-alkyl stands for one of the abovementioned 1-4C-alkyl radicals, which is substituted by one of the abovementioned 1-4C-alkoxycarbonyl radicals. An example is the ethoxycarbonylmethyl radical [CH₃CH₂OC(O)CH₂-].

1-4C-Alkoxy-2-4C-alkyl represents a 2-4C-alkyl radical, which is substituted by one of the abovementioned 1-4C-alkoxy radicals. Examples which may be mentioned are the methoxyethyl and the ethoxyethyl radical.

Phenyl-1-4C-alkyl radicals stand for one of the abovementioned 1-4C-alkyl radicals substituted by an phenyl group. Examples which may be mentioned are the phenylethyl and the benzyl radical.

R29(R30)N-2-4C-alkyl radicals stand for one of the above-mentioned 2-4C-radicals substituted by an R29(R30)N- group. Examples which may be mentioned are morpholin-4-ylethyl and the thiomorpholin-4-ylethyl radicals.

“N-oxides of these compounds” stands for any single or multiple N-oxide(s), which can be formed starting from the compounds of formula 1. Preferred are the single N-oxides at the nitrogen atom in 5-position of the phenanthridine ring system.

In the formulae (a), (b), (c) or (d) the horizontal dotted lines indicate 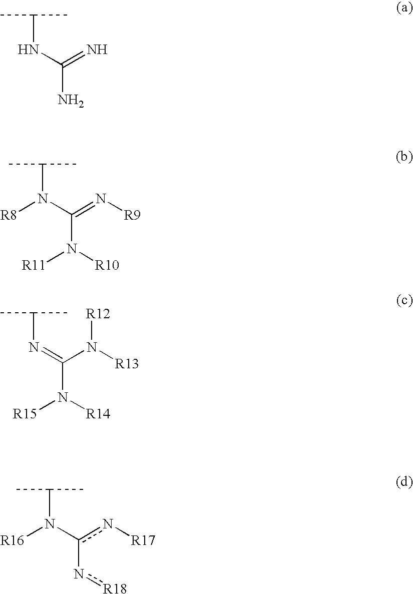
that R7 is bonded to the carbonyl group in formula 1 via the bond that bears the horizontal dotted line. The additional dotted lines in formula (d) indicate that there can be in the indicated positions a single or a double bond.

The substituents R6 and —C(O)R7 of the compounds of the formula 1 can be attached in the ortho, meta or para position with respect to the binding position in which the 6-phenyl ring is bonded to the phenanthridine ring system. Preference is given to compounds of the formula 1, in which R6 is hydrogen and —C(O)R7 is attached in the meta or in the para position.

Suitable salts of compounds of the formula 1—depending on substitution—are all acid addition salts or all salts with bases. The pharmacologically tolerable salts of the inorganic and organic acids and bases customarily used in pharmacy may be particularly mentioned. Those suitable are, on the one hand, water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, where the acids are employed in salt preparation—depending on whether a mono- or polybasic acid is concerned and depending on which salt is desired—in an equimolar quantitative ratio or one differing therefrom.

On the other hand salts with bases are also suitable. Examples of salts with bases which may be mentioned are alkali metal (lithium, sodium, potassium) or calcium, aluminum, magnesium or titanium salts, where here too the bases are employed in salt preparation in an equimolar quantitative ratio or one differing therefrom.

Pharmacologically intolerable salts which can be obtained first, for example, as process products in the preparation of the compounds according to the invention on an industrial scale, are converted into pharmacologically tolerable salts by methods known to the person skilled in the art

It is known to the person skilled in the art that the compounds according to the invention and their salts, for example when they are isolated in crystalline form, may comprise varying amounts of solvents. Accordingly, the invention also embraces all solvates and in particular all hydrates of the compounds of the formula 1, and also all solvates and in particular all hydrates of the salts of the compounds of the formula 1.

Compounds of the formula 1 to be emphasized are those in which
R1 is hydroxyl, 1-4C-alkoxy, 3-7C-cycloalkoxy, 3-7C-cycloalkylmethoxy or completely or predominantly fluorine-substituted 1-4C-alkoxy, and
R2 is hydroxyl, 1-4C-alkoxy, 3-7C-cycloalkoxy, 3-7C-cycloalkylmethoxy or completely or predominantly fluorine-substituted 1-4C-alkoxy,
R1 and R2 together are a 1-2C-alkylenedioxy group,
R3 is hydrogen or 1-4C-alkyl,
R31 is hydrogen or 1-4C-alkyl,
or in which
R3 and R31 together are a 1-4C-alkylene group,
R4 is hydrogen or 1-4C-alkyl,
R5 is hydrogen,
R51 is hydrogen,
or in which
R5 and R51 together represent an additional bond,
R6 is hydrogen, halogen, nitro, 1-4C-alkyl, trifluoromethyl or 1-4C-alkoxy,
R7 is a radical of formulae (a), (b), (c) or (d) 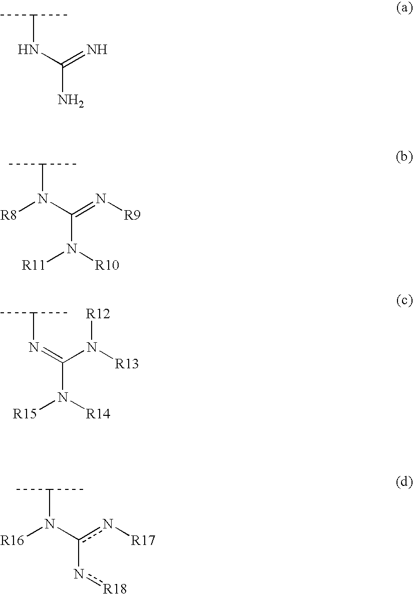
   in which
if R7 is a radical of the formula (b),
either
   R8, R9, R10 and R11 independently of one another are hydrogen, 1-7C-alkyl, 3-7C-cycloalkyl, 3-7C-cycloalkylmethyl or hydroxy-2-4C-alkyl,
      or
   R8 is hydrogen, 1-7C-alkyl, 3-7C-cycloalkyl, 3-7C-cycloalkylmethyl or hydroxy-2-4C-alkyl, R9 is hydrogen, 1-7C-alkyl, 3-7C-cycloalkyl, 3-7C-cycloalkylmethyl or hydroxy-2-4C-alkyl, and R10 and R11, together and including the nitrogen atom to which both are bonded, are a pyrrolidin-1-yl, piperidin-1-yl, azepan-1-yl, azocan-1-yl, azonan-1-yl, azecan-1-yl, morpholin-4-yl, tetrahydroisoquinolin-2-yl, 3,5-dimethyl-pyrazol-1-yl, pyrazol-1-yl, 2,6-dimethyl-morpholin-4-yl, 2,6-dimethyl-piperidin-1-yl, thiomorpholin-4-yl or 1H-1,2,4-triazol-1-yl radical, or a piperazin-1-yl radical substituted in 4-position by R19,
      or
   R8 is hydrogen, 1-7C-alkyl, 3-7Cycloalkyl, 3-7C-cycloalkylmethyl or hydroxy-2-4C-alkyl,
   R9 is hydrogen, 1-7C-alkyl; 3-7C-cycloalkyl, 3-7C-cycloalkylmethyl or hydroxy-2-4C-alkyl,
   R10 is hydrogen, 1-7C-alkyl, 3-7C-cycloalkyl, 3-7C-cycloalkylmethyl or hydroxy-2-4C-alkyl, and
   R11 is Aryl1, naphthyl, phenyl, phenyl substituted by R20 and/or R21, phenyl-1-4C-alkyl or phenyl-1-4C-alkyl substituted by R22 and R23,
      in which
if R7 is a radical of the formula (c),
either
   R12, R13, R14 and R15 independently of one another are hydrogen,. 1-7C-alkyl, 3-7C-cycloalkyl, 3-7C-cycloalkylmethyl or hydroxy-2-4C-alkyl,
      or
   R12 and R13 independently of one another are hydrogen, 1-7C-alkyl, 3-7C-cycloalkyl, 3-7C-cycloalkylmethyl or hydroxy-2-4C-alkyl, and
   R14 and R15, together and including the nitrogen atom to which both are bonded, are a pyrrolidin-1-yl, piperidin-1-yl, azepan-1-yl, azocan-1-yl, azonan-1-yl, azecan-1-yl, morpholin-4-yl, tetrahydroisoquinolin-2-yl, 3,5-dimethyl-pyrazol-1-yl, pyrazol-1-yl, 2,6-dimethyl-morpholin4yl, 2,6-dimethyl-piperidin-1-yl, thiomorpholin-4-yl or 1H-1,2,4-triazol-1-yl radical, or a piperazin-1-yl radical substituted in 4-position by R19,
      or

R12 and R13, together and including the nitrogen atom to which both are bonded, are a pyrrolidin-1-yl, piperidin-1-yl, azepan-1-yl, morpholino-4-yl, 4-(1-4C-alkyl-)-piperazin-1-yl, 2,6-dimethylmorpholin-4-yl, 2,6-dimethyl-piperidin-1-yl or thiomorpholin-4-yl radical, and R14 and R15, together and including the nitrogen atom to which both are bonded, are a pyrrolidin-1-yl, piperidin-1-yl, azepan-1-yl, morpholino-4-yl, 4-(1-4C-alkyl-)-piperazin-1-yl, 2,6-dimethylmorpholin-4-yl, 2,6-dimethyl-piperidin-1-yl or thiomorpholin-4-yl radical,

or

R12 and R15 independently of one another are hydrogen or 1-4C-alkyl, and
R13 and R14, together and with inclusion of the N—C(═)—N structure to which they are bonded, are a hexahydropyrimidin-2-ylidene or imidazolidin-2-ylidene radical,
   in which
if R7 is a radical of the formula (d),
   R16 is hydrogen, 1-7C-alkyl, 3-7C-cycloalkyl, 3-7C-cycloalkylmethyl or hydroxy-2-4C-alkyl, and
   R17 and R18, together and with inclusion of the N—C(—)—N structure to which they are bonded are Aryl2,
   Aryl1 is 4-methylthiazol-2-yl, benzimidazol-2-yl, 5-nitrobenzimidazol-2-yl, 5-chlorobenzimidazol-2-yl, 5-methylbenzimidazol-2-yl, 4-methylquinazolin-2-yl, benzothiazol-2-yl, benzoxazol-2-yl or pyrimidin-2-yl,
   Aryl2 is 1-methyl-4-oxo-4,5-dihydro-1H-imidazol-2-yl, imidazol-2-yl, 4,5-dicyano-imidazol-2-yl, 4-methyl-imidazol-2-yl; 4-ethyl-benzimidazol-2-yl, 4-acetyl-imidazol-2-yl, 1H-[1,2,4]triazol-3-yl, benzimidazol-2-yl, 1-methyl-benzimidazol-2-yl, 1-ethyl-benzimidazol-2-yl, 5,6-dimethyl-benzimidazol-2-yl, purin-8-yl, 6-amino-7-methyl-7H-purine-8-yl, 1,6-dimethylimidazo[4,5-b]pyridin-2-yl, 1,5,6-trimethylimidazo[4,5-b]pyridin-2-yl, 1,3-dimethyl-3,7-dihydro-1H-purine-2,6-dione8-yl, 7-ethyl-3-methyl-3,7-dihydro-purine-2,6-dione-8-yl, 1,3,7-trimethyl-3,7-dihydro-purine-2,6-dione-8-yl, thiadiazolyl, 1,4-dihydrotetrazol-5-yl, 2H-[1,2,4]triazol-3-yl, 1,3-dihydrobenzimidazol-5-yl, 1H-tetrazol-5-yl, pyrimidin-2-yl or 4,6-dimethyl-pyrimidin-2-yl,
R19 is 1-4C-alkyl, formyl, 1-4C-alkylcarbonyl, 2-hydroxyethyl, phenyl, phenyl substituted by R24 and/or R25, phenyl-1-4C-alkyl or phenyl-1-4C-alkyl substituted in the phenyl moiety by R26 and/or R27,
R20 is-halogen, nitro, carboxyl, 1-4C-alkyl, trifluoromethyl or 1-4C-alkoxy,
R21 is halogen, 1-4C-alkyl or 1-4C-alkoxy,
R22 is halogen, nitro, carboxyl, 1-4C-alkyl, trifluoromethyl or 1-4C-alkoxy,
R23 is halogen, 1-4C-alkyl or 1-4C-alkoxy,
R24 is halogen, nitro, carboxyl, 1-4C-alkyl, trifluoromethyl or 1-4C-alkoxy,
R25 is halogen, 1-4C-alkyl or 1-4C-alkoxy,
R26 is halogen, nitro, carboxyl, 1-4C-alkyl, trifluoromethyl or 1-4C-alkoxy,
R27 is halogen, 1-4C-alkyl or 1-4C-alkoxy, the salts of these compounds, as well as the N-oxides, enantiomers, E/Z isomers and tautomers of these compounds and their salts.

Compounds of the formula 1 to be particularly emphasized are those in which
R1 is 1-2C-alkoxy, 3-5C-cycloalkoxy, 3-5C-cycloalkylmethoxy or completely or predominantly fluorine-substituted 1-2C-alkoxy,
R2 is 1-2C-alkoxy, 3-5C-cycloalkoxy, 3-5Cycloalkylmethoxy or completely or predominantly fluorine-substituted 1-2C-alkoxy,
R3 is hydrogen,
R31 is hydrogen,
R4 is hydrogen or 1-2C-alkyl,
R5 is hydrogen,
R51 is hydrogen,
or in which
R5 and R51 together represent an additional bond,
R6 is hydrogen, halogen, nitro, 1-4C-alkyl, trifluoromethyl or 1-4C-alkoxy,
R7 is a radical of formulae (a), (b), (c) or (d) 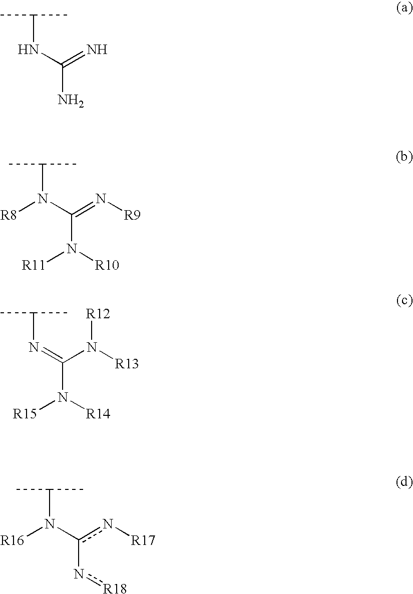
   in which
if R7 is a radical of the formula (b),
either
   R8 is hydrogen, and
   R9, R10 and R11 independently of one another are hydrogen, 1-4C-alkyl, 3-7C-cycloalkyl or 3-7C-cycloalkylmethyl,
or
   R8 is hydrogen,
   R9 is hydrogen, 1-4C-alkyl, 3-7C-cycloalkyl or 3-7C-cycloalkylmethyl, and
   R10 and R11, together and including the nitrogen atom to which both are bonded, are a pyrrolidin-1-yl, piperidin-1-yl, azepan-1-yl, azocan-1-yl, azonan-1-yl, azecan-1-yl, morpholin-4-yl, tetrahydroisoquinolin-2-yl, 3,5-dimethyl-pyrazol-1-yl, pyrazol-1-yl, 2,6-dimethyl-morpholin-4-yl or 2,6-dimethyl-piperidin-1-yl radical, or a piperazin-1-yl radical substituted in 4-position by R19,
or
   R8 is hydrogen,
   R9 is hydrogen, 1-7C-alkyl, 3-7C-cycloalkyl or 3-7C-cycloalkylmethyl,
   R10 is hydrogen, 1-7C-alkyl, 3-7C-cycloalkyl or 3-7C-cycloalkylmethyl, and
   R11 is Aryl1, naphthyl, phenyl, phenyl substituted by R20 and/or R21, phenyl-1-4C-alkyl or phenyl-1-4C-alkyl substituted by R22 and R23,
      in which
if R7 is a radical of the formula (c),
either
   R12, R13, R14 and R15 independently of one another are hydrogen, 1-4C-alkyl, 3-7C-cycloalkyl or 3-7C-cycloalkylmethyl,
or
   R12 and R13 independently of one another are hydrogen, 1-4C-alkyl, 3-7C-cycloalkyl or 3-7C-cycloalkylmethyl, and
   R14 and R15, together and including the nitrogen atom to which both are bonded, are a pyrrolidin-1-yl, piperidin-1-yl, azepan-1-yl, azocan-1-yl, azonan-1-yl, azecan-1-yl, morpholin-4-yl, tetrahydroisoquinolin-2-yl, 3,5-dimethyl-pyrazol-1-yl, pyrazol-1-yl, 2,6-dimethyl-morpholin-4-yl or 2,6-dimethyl-piperidin-1-yl radical, or a piperazin-1-yl radical substituted in 4 position by R19,
or
   R12 and R13, together and including the nitrogen atom to which both are bonded, are a pyrrolidin-1-yl, piperidin-1-yl, azepan-1-yl, morpholino-4-yl, 4(1-4C-alkyl-)-piperazin-1-yl, 2,6-dimethylmorpholin-4-yl or 2,6-dimethyl-piperidin-1-yl radical, and
   R14 and R15, together and including the nitrogen atom to which both are bonded, are a pyrrolidin-1-yl, piperidin-1-yl, azepan-1-yl, morpholino-4yl, 4(1-4C-alkyl-)-piperazin-1-yl, 2,6-dimethylmorpholin-4-yl or 2,6-dimethyl-piperidin-1-yl radical, and
or
   R12 and R15 independently of one another are hydrogen or 1-4C-alkyl, and
   R13 and R14, together and including the N—C(═)—N structure to which they are bonded, are a hexahydropyrimidin-2-ylidene or imidazolidin-2-ylidene radical,
in which
if R7 is a radical of the formula (d),
   R16 is hydrogen, and
   R17 and R18, together and with inclusion of the N—C(—)—N structure to which they are bonded are Aryl 2,
Aryl1 is 4-methylthiazol-2-yl, benzimidazol-2-yl, 5-nitrobenzimidazol-2-yl, 5-chlorobenzimidazol-2-yl, 5-methylbenzimidazol-2-yl, benzothiazol-2-yl or benzoxazol-2-yl,
   Aryl 2 is 1-methyl-4-oxo-4,5-dihydro-1H-imidazol-2-yl, imidazol-2-yl, 4,5dicyano-imidazol-2-yl, 4-methyl-imidazol-2-yl, 4-ethyl-benzimidazol-2-yl, 4-acetyl-imidazol-2-yl, 1H-[1,2,4]triazol-3-yl, benzimidazol-2-yl, 1-methyl-benzimidazol-2-yl, 1-ethyl-benzimidazol-2-yl, 5,6-dimethyl-benzimidazol-2-yl, purin-8-yl, 6-amino-7-methyl-7H-purine-8-yl, 1,6-dimethylimidazo[4,5-b]pyridin-2-yl, 1,5,6-trimethylimidazo[4,5-b]pyridin-2-yl, 1,3-dimethyl-3,7-dihydro-1H-purine-2,6-dione-8-yl, 7-ethyl3-methyl-3,7-dihydro-purine-2,6-dione-8-yl, 1,3,7-trimethyl-3,7-dihydro-purine-2,6-dione-8-yl or 1H-[1,2,4]triazol-3-yl,
R19 is 1-4C-alkyl, formyl, 1-4C-alkylcarbonyl, 2-hydroxyethyl, phenyl, phenyl substituted by R24 and/or R25, phenyl-1-4C-alkyl or phenyl-1-4C-alkyl substituted in the phenyl moiety by R26 and/or R27,
R20 is halogen, nitro, 1-4C-alkyl or 1-4C-alkoxy,
R21 is halogen, 1-4C-alkyl or 1-4C-alkoxy,
R22 is halogen, nitro, 1-4C-alkyl or 1-4C-alkoxy,
R23 is halogen, 1-4C-alkyl or 1-4C-alkoxy,
R24 is halogen, nitro, 1-4C-alkyl or 1-4C-alkoxy,
R25 is halogen, 1-4C-alkyl or 1-4C-alkoxy,
R26 is halogen, nitro, 1-4C-alkyl or 1-4C-alkoxy,
R27 is halogen, 1-4C-alkyl or 1-4C-alkoxy,
the salts of these compounds, as well as the N-oxides, enantiomers, E/Z isomers and tautomers of these compounds and their salts.

Preferred compounds of the formula 1 are those in which
R1 is 1-2C-alkoxy,
R2 is 1-2C-alkoxy,
R3, R31, R4, R5 and R51 are hydrogen,
R6 is hydrogen,
R7 is a radical of formulae (a), (b), (c) or (d) 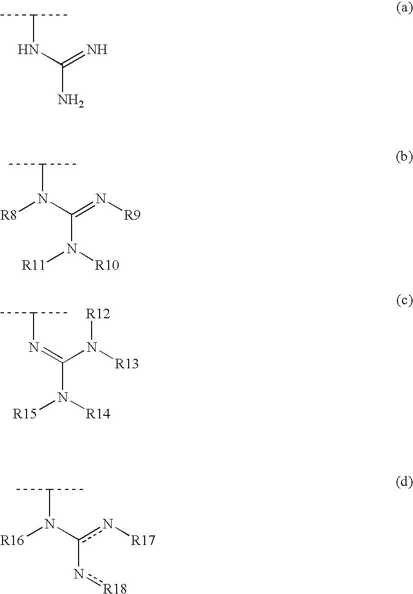
   in which
if R7 is a radical of the formula (b),
either
   R8 is hydrogen,
   R9 is hydrogen,
   R10 is hydrogen or 1-4C-alkyl,
   R11 is hydrogen or 1-4C-alkyl,
   where at least one of the radicals R10 or R11 is not hydrogen,
or
   R8 is hydrogen,
   R9 is hydrogen,
   R10 and R11, together and including the nitrogen atom to which both are bonded, are a pyrrolidin-1-yl, piperidin-1-yl, azepan-1-yl, azocan-1-yl, azonan-1-yl, morpholin-4-yl, tetrahydroisoquinolin-2-yl or 3,5-dimethyl-pyrazol-1-yl radical, or a piperazin-1-yl radical substituted by R19,
or
   R8 is hydrogen,
   R9 is hydrogen,
   R10 is hydrogen or 1-4C-alkyl, and
   R11 is Aryl1, naphthyl, phenyl or phenyl substituted by R20,

in which
if R7 is a radical of the formula (c),
either
   R12 is hydrogen or 1-4C-alkyl,
   R13 is hydrogen or 1-4C-alkyl,
   R14 is hydrogen or 1-4C-alkyl, and
   R15 is hydrogen or 1-4C-alkyl,
   where at least one of the radicals R12, R13, R14 and R15 is not hydrogen,
or
   R12 is hydrogen or 1-4C-alkyl,
   R13 is hydrogen or 1-4C-alkyl, and
   R14 and R15, together and including the nitrogen atom to which both are bonded, are a pyrrolidin-1-yl, piperidin-1-yl, azepan-1-yl, azocan-1-yl, azonan-1-yl, morpholin-4-yl, tetrahydroisoquinolin-2-yl or 3,5-dimethyl-pyrazol-1-yl radical, or a piperazin-1-yl radical substituted by R19,

in which
if R7 is a radical of the formula (d),
   R16 is hydrogen, and
   R17 and R18, together and with inclusion of the N—C(—)—N structure to which they are bonded are Aryl 2,
Aryl1 is benzimidazol-2-yl, 5-nitrobenzimidazol-2-yl, 5-chlorobenzimidazol-2-yl or 5-methylbenzimidazol-2-yl,
Aryl2 is imidazol-2-yl, 4-methyl-imidazol-2-yl, 4-ethyl-benzimidazol-2-yl, 4-acetyl-imidazol-2-yl, 1H-[1,2,4]triazol-3-yl, benzimidazol-2-yl, 1-methyl-benzimidazol-2-yl, 1-ethyl-benzimidazol-2-yl, 5,6-dimethyl-benzimidazol-2-yl, purin-8-yl, 1,6-dimethylimidazo[4,5-b]pyridin-2-yl, 1,5,6-trimethylimidazo[4,5-b]pyridin-2-yl,
R19 is 1-4C-alkyl,
R20 is halogen, nitro, 1-4C-alkyl or 1-4C-alkoxy,
the salts of these compounds, as well as the N-oxides, enantiomers, E/Z isomers and tautomers of these compounds and their salts.

Particularly preferred compounds of the formula 1 are those in which
R1 is methoxy,
R2 is methoxy,
R3, R31, R4, R5 and R51 are hydrogen,
R6 is hydrogen,
R7 is a radical selected from 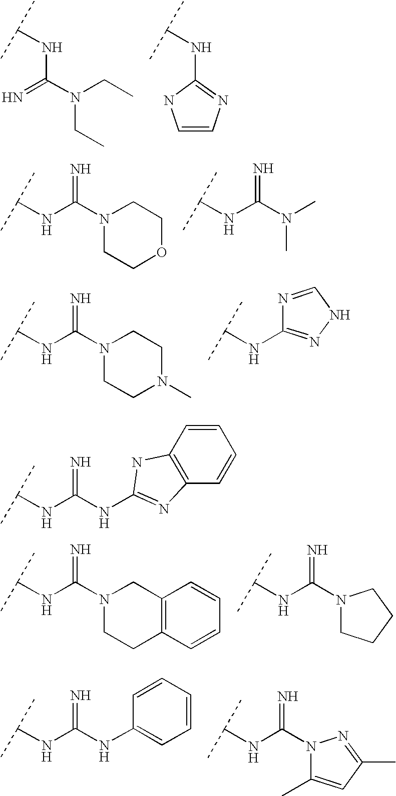
   the salts of these compounds, as well as the N-oxides, enantiomers, E/Z isomers and tautomers of these compounds and their salts.

A special embodiment of the compounds of the present invention include those compounds of formula 1 in which R1 and R2 are 1-2C-alkoxy.

Another special embodiment of the compounds of the present invention include those compounds of formula 1 in which R1 and R2 are 1-2C-alkoxy and R3, R31, R4, R5 and R51 are hydrogen.

Still another special embodiment of the compounds of the present invention include those compounds of formula 1 in which R1 and R2 are 1-2C-alkoxy and R3, R31, R4, R5, R51 and R6 are hydrogen.

A further special embodiment of the compounds of the present invention include those compounds of formula 1 in which R1 and R2 are 1-2C-alkoxy, R3, R31, R4, R5, R51 and R6 are hydrogen and R7 is a radical of formulae (b) or (c).

Another further special embodiment of the compounds of the present invention include those compounds of formula 1 in which R1 and R2 are 1-2C-alkoxy, R3, R31, R4, R5, R51 and R6 are hydrogen and R7 is a radical of formula (d).

The compounds of formula 1 are chiral compounds having chiral centers at least in positions 4a and 10b and depending on the meanings of R3, R31, R4, R5 and R51 additional chiral centers in positions 1, 2, 3 and 4.

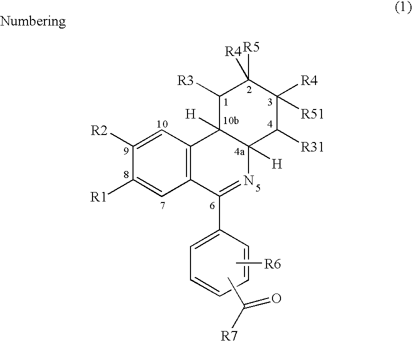

The invention includes all conceivable stereoisomers in pure form as well as in any mixing ratio. Preference is given to compounds of formula 1 in which the hydrogen atoms in positions 4a and 10b are in the cis position relative to one another. The pure cis enantiomers and their mixtures in any mixing ratio and including the racemates are particularly preferred.

Particularly preferred in this context are those compounds of formula 1, which have with respect to the positions 4a and 10b the configuration shown in formula (1*):

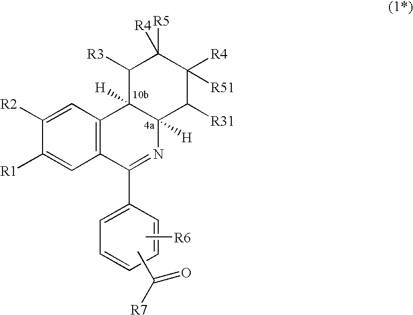

If, for example, in compounds of formula 1* R3, R31, R4, R5 and R51 have the meaning hydrogen, then the configuration—according to the rules of Cahn, Ingold and Prelog—is R in the 4a position and R in the 10b position.

The enantiomers can be separated in a manner known per se (for example by preparation and separation of appropriate diastereoisomeric compounds). Preferably, an enantiomer separation is carried out at the stage of the staring compounds of formula 7 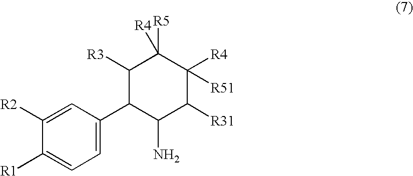
for example by means of salt formation of the racemic compounds of formula 7 with optically active carboxylic acids. Examples which may be mentioned in this connection are the enantiomeric forms of mandelic acid, tartaric acid, O,O′-dibenzoyltartaric acid, camphoric acid, quinic acid, glutamic acid, malic acid, camphorsulfonic acid, 3-bromocamphorsulfonic acid, α-methoxyphenylacetic acid, α-methoxy-α-trifluoromethylphenylacetic acid and 2-phenylpropionic acid. Alternatively, enantiomerically pure starting compounds of formula 7 can also be prepared via asymmetric syntheses.

The compounds according to the invention can be prepared, for example, as shown in the reaction schemes below.

Reaction scheme 1: In a first reaction step, compounds of formula 7, in which R1, R2, R3, R31, R4, R5 and R51 have the meanings given above, are reacted with compounds of formula 6, in which R6 has the meanings given above, R is, for example, 1-4C-alkyl and X is a suitable leaving group, for example a chlorine atom. The benzoylation is carried out, for example, according to the Einhorn process, the Schotten-Baumann variant or as described in J. Chem. Soc. C, 1971, 1805-1808.

The compounds of formula 4 are obtained by cyclocondensation of the compounds of formula 5.

The cyclocondensation is carried out in a manner known per se to the person skilled in the art, according to Bischler-Napieralski (e.g. as described in J. Chem. Soc., 1956, 4280-4282) in the presence of a suitable condensing agent such as, for example, polyphosphoric acid, phosphorus pentachloride, phosphorus pentoxide or preferably phosphorus oxychloride, in a suitable inert solvent, e.g. in a chlorinated hydrocarbon such as chloroform, or in a cyclic hydrocarbon such as toluene or xylene, or another inert solvent such as acetonitrile, or without further solvent using an excess of condensing agent, preferably at elevated temperature, in particular at the boiling temperature of the solvent or condensing agent used.

Starting with the compounds of formula 4, the compounds of formula 1 can be obtained by different routes. On the one hand, the compounds of formula I can be obtained from the compounds of formula 4 by direct reaction with compounds of formula R7-H, in which R7 has the meanings given above.

On the other hand the compounds of formula 4 can be first saponified to give the benzoic acid derivatives of formula 3, which then can be activated prior to the reaction with compounds of formula R7-H for example by forming an acid halide or acid anhydride, or by using coupling agents known to the person skilled in the art, such as, for example, N,N′-dicyclohexylcarbodiimide or N′-(3-dimethylaminopropyl)—N-ethylcarbodiimide (compounds of formula 2).

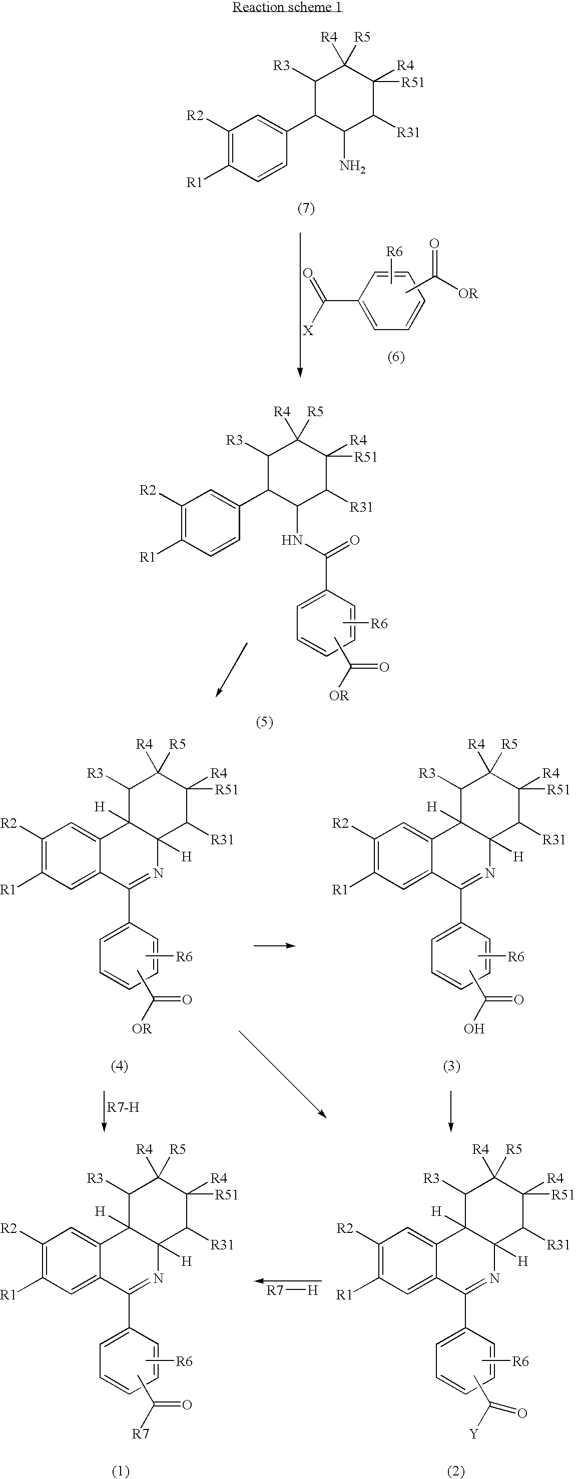

It is also possible to obtain compounds of formula 1 from compounds of formula 2 by initially reacting the compounds of formula 2 in which Y is, for example, a chlorine atom with suitably substituted S-alkyl-isothioureas and then, in a second step, replacing the S-alkyl group by a suitably substituted amine.

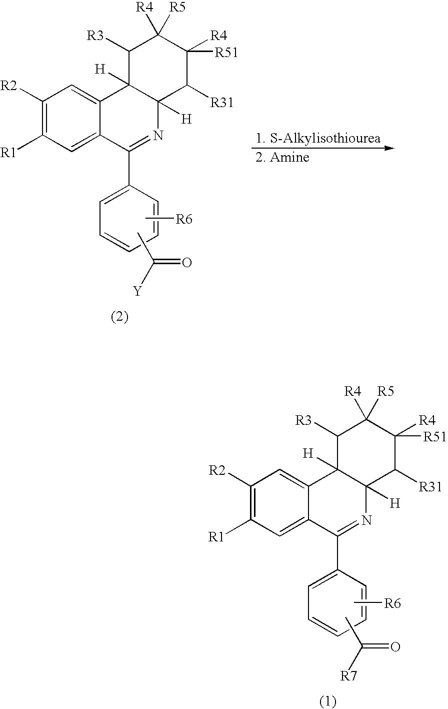

Similar reactions are described, for example in Arzneim.-Forsch. (Drug Res.) 25, No. 10, (1975), pp. 1477-1482 or in the following examples.

The preparation of compounds of formula 4, in which R1, R2, R3, R4, R5, R51 and R6 have the meanings given above and R is 1-4C-alkyl as well as of benzoic acid derivatives of formula 3, in which R1, R2, R3, R4₇ R5, R51 and R6 have the meanings given above, is described in the international application WO97/28131 and in the following examples.

Compounds of the formula 6 are known or can be prepared according to known processes such as, for example, the process shown in reaction scheme 2. The preparation of pure enantiomeres of compounds of formula 7 is described, for example, in the international application WO00/42020 and in the following examples.

An alterative synthesis route for compounds of formula 1 is shown in reaction scheme 2.

Starting with a suitably substituted phthalic acid, isophthalic acid or terephthalic acid monoester derivative (compounds of formula 12), the acid group is initially activated, for example by forming an acid halide (compounds of formula 6).

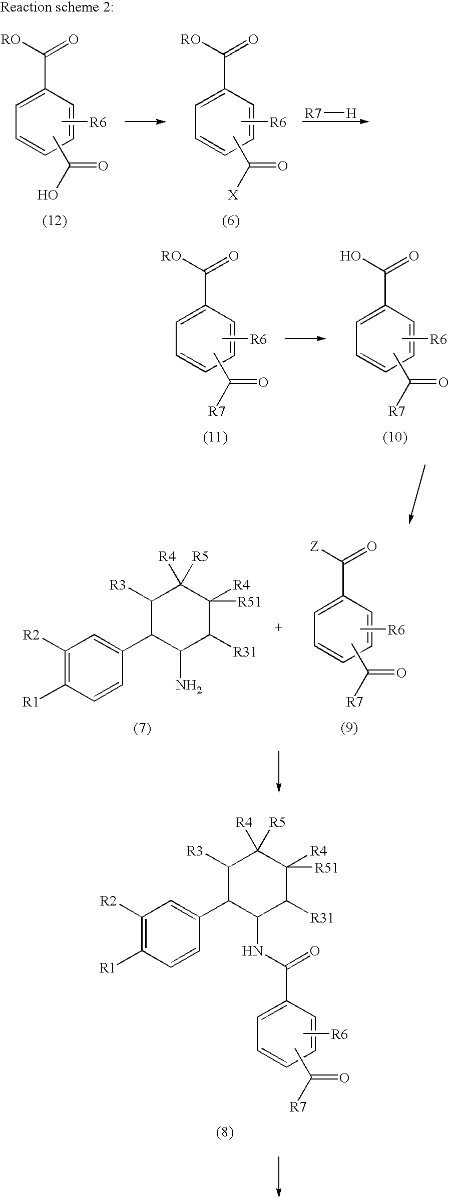

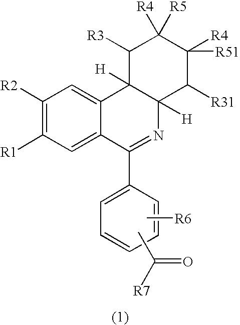

The acid halide (compounds of formula 6) is then reacted with compounds of formula R7-H, in which R7 has the meanings given above. The ester group of the resulting guanidine derivatives (compounds of formula 11) is hydrolyzed and the resulting acids (compounds of formula 10) are activated, for example by conversion into an acid halide (Z for example Cl; compounds of formula 9).

In the next reaction step, compounds of formula 7, in which R1, R2, R3,R31, R4, R5 and R51 have the meanings given above are benzoylated with the compounds of formula 9. Again, this benzoylation is carried out, for example, by the Einhorn process, the Schotten-Baumann variant or as described in J. Chem. Soc. (C), 1971, 1805-1808.

The final cyclocondensation of the compounds of formula 8 obtained by the benzoylation affords the compounds of formula 1.

The compounds of formula 1 prepared by the processes described above can then, if desired, be converted into their salts, or salts of the compounds of formula 1 obtained can then, if desired, be converted into the free compounds. Corresponding processes are known to the person skilled in the art.

Suitably substituted phthalic acid, isophthalic acid or terephthalic acid monoester derivatives (compounds of formula 6 or 12) are either known or can be prepared by methods known to the person skilled in the art Exemplary compounds of formula 6 which may be mentioned are methyl 4-chlorocarbonylbenzoate (preparation described in J. Amer. Chem. Soc. 79, (1957), 96 or in Bioorg. Med. Chem. Lett. 1999, 227-232) and methyl 3-chlorocarbonylbenzoate (preparation described in J. Med. Chem. 1999, 2621-2632).

In addition, the compounds of formula I can be converted by derivatisation into further compounds of formula 1. Thus, for example, compounds of formula 1 can be converted, if desired, into their N-oxides.

The N-oxidation is carried out in a manner which is known to the person skilled in the art, for example with the aid of hydrogen peroxide in methanol or with the aid of m-chloroperoxybenzoic acid in dichloromethane. The person skilled in the art is familiar on the basis of his/her expert knowledge with the reaction conditions which are specifically necessary for carrying out the N-oxidation.

It is also known to the person skilled in the art that, if a plurality of reactive centers are present in a starting material or intermediate, it may be necessary to temporarily block one or more reactive centers with protective groups so that a reaction takes place only at the desired reactive center. A detailed description of how to use a large number of proven protective groups can be found, for example, in T.W. Greene, Protective Groups in Organic Synthesis, John Wiley & Sons, 1991.

The substances according to the invention are isolated and purified in a manner known per se, for example by distilling off the solvent under reduced pressure and recrystallizing the residue obtained from a suitable solvent or subjecting it to one of the customary purification methods, such as, for example, column chromatography on a suitable support material.

Salts are obtained by dissolving the free compound in a suitable solvent (e.g. a ketone, such as acetone, methyl ethyl ketone or methyl isobutyl ketone, an ether, such as diethyl ether; tetrahydrofuran or dioxane, a chlorinated hydrocarbon, such as methylene chloride or chloroform, or a low-molecular-weight aliphatic alcohol, such as ethanol or isopropanol) which contains the desired acid or base, or to which the desired acid or base is then added. The salts are obtained by filtering, reprecipitating, precipitating with a nonsolvent for the addition salt or by evaporating the solvent Salts obtained can be converted into the free compounds, which can in turn be converted into salts, by alkalization or by acidification. In this manner, pharmacologically unacceptable salts can be converted into pharmacologically acceptable salts.

The following examples serve to illustrate the invention in greater detail without restricting it Further compounds of the formula 1, whose preparation is not explicitly described, can also be prepared in an analogous manner or in a manner familiar per se to the person skilled in the art using customary process techniques.

In the examples, m.p. stands for melting point, h for hour(s), RT for room temperature, calc for calculated and fnd for found. The compounds mentioned in the examples and their salts are preferred subject of the invention.

### EXAMPLES

End Products

1. N′-{-[4-((4aR,10bR)-8,9-Dimethoxy-1,2,3,4,4a,10b-hexahydrophenanthridin-6yl)-phenyl]-methanoyl}-N,N-diethylquanidine

4.9 g 1,1-Diethylguanidiniumsulfat are suspended in 120 ml acetonitrile. To this solution 720 mg sodium hydroxide are added in 25 ml methanol. After stirring for 1 h at RT the solvent is evaporated, the residue is suspended in 200 ml dichloromethane and 5.2 g of sodium carbonate are added. A solution of 4.2 g 4-((4aR,10bR)-8,9-Dimethoxy-1,2,3,4,4a,10b-hexahydrophenanthridin-6-yl)-benzoylchloride hydrochloride in 200 ml dichloromethane is added dropwise and the resulting mixture is stirred for 15 h at RT. Then the reaction mixture is extracted with 1 M hydrochloric acid; the aqueous phase is made basic with 10 M sodium hydroxide solution and extracted with dichloromethane. The aqueous phase is dried over sodium sulfate and the solvent is evaporated. The residue is purified by chromatography (silica gel;toluene/ethyl acetate/triethyl amine=5/3/1). 640 mg of the title compound are obtained. M.p. 132-135° C.
MS: calc.: C₂₇H₃₄N₄O₃(462,6) fnd.: [M+1] 463,2

Analogously to example 1, the following title compounds are obtained when, instead of N,N-diethylguanidiniumsulfate, the respective appropriately substituted guanidines are used as reaction partners:

2. 4-((4aR.10bR)8,9-Dimethoxy-1,2,3,4,4a.10b-hexahydrophenanthridin-6-yl)-N-(1H-imidazol-2-yl)-benzamide

MS: calc.: C₂₅H₂₆N₄O₃(430,51) fnd.: [M+1] 431,4
   3. 4(4aR.10bR)-8,9-Dimethoxy-1,2,3,4,4a.10b-hexahydrophenanthridin-6-yl)-N-(1-imino-1-morpholin-4-yl-methyl)-benzamide
M.p. 136° C.
MS: calc.: C₂₇H₃₂N₄O₄(476,58) fnd.: [M+1] 477,1
   4. N′-{1-[4-((4aR,10bR)-8,9-Dimethoxy-1,2,3,4,4a,10b-hexahydrophenanthridin-6-yl)-phenyl]-methanoyl}-N,N-dimethylquanidine
M.p. 185° C.
MS: calc.: C₂₅H₃₀N₄O₃(434,54) fnd.: [M+1] 435,1:
   5. 4((4aR,10bR)-8,9-Dimethoxy-1,2,3,4,4a,10b-hexahydro-phenanthridin-6-yl)-N-[1-imino-2-4-methyl-piperazin-1-yl)-methyl]-benzamide
M.p. 133° C.
MS: calc.: C₂₈H₃₅N₅O₃(489,62) fnd.: [M+1] 490,2
   6. 4-((4aR,10bR)-8,9-Dimethoxy-1,2,3,4,4a,10b-hexahydro-phenanthridin-6-yl)-N-(1H-[1,2,4]triazol-3-yl)-benzamide
MS: calc.: C₂₄H₂₅N₅O₃(431,5) fnd.: [M+1] 432,4
   7. N-(1H-Benzoimidazol-2-yl)-N′-{1-[4-((4aR,10bR)-8,9-dimethoxy-1,2,3,4,4a,10b-hexahydro-phenanthridin-6-yl)-phenyl]-methanoyl}-quanidine
MS: calc.: C₃₀H₃₀N₆O₃(522,61) fnd.: [M+1] 523,2
   8. N-[1-(Tetrahydroisoquinolin-2-yl)-1imino-methyl]-4-((4aR,10bR)-8,9-dimethoxy-1,2,3,4,4a,10b-hexahydro-phenanthridin-6-yl)-benzamide
MS: calc.: C₃₂H₃₄N₄O₃(522,65) fnd.: [M+1] 523,2
   9. 4-((4aR,10bR)-8,9-Dimethoxy-1,2,3,4,4a.10b-hexahydro-phenanthridin-6-yl)-N-(1-imino-1-pyrrolidin-1-yl-methyl)-benzamide
M.p. 126° C.
MS: calc.: C₂₇H₃₂N₄O₃(460,58) fnd.: [M+1] 461,2
   10. N{1-[4-((4aR,10bR)-8,9-Dimethoxy-1,2,3,4,4a,10b-hexahydro-phenanthridin-6-yl)-phenyl]-methanoyl}-N′-phenyl-quanidine
MS: calc.: C₂₉H₃₀N₄O₃(482,59) fnd.: [M+1] 483,2
   11. 4((4aR,10bR)-8,9-Dimethoxy-1,2,3,4,4a,10b-hexahydro-phenanthridin-6-yl)-N-[1-(3,5-dimethyl-pyrazol-1-yl)-1-imino-methyl]-benzamide
M.p. 179-180° C.
MS: calc.: C₂₈H₃₁N₅O₃(485,59) fnd.: [M+1] 485,9

Analogously to example 1, the following title compounds are obtained when instead of 4-((4aR,10bR)-8,9-Dimethoxy-1,2,3,4,4a,10b-hexahydrophenanthridin6-yl)-benzoylchloride hydrochloride 3-((4aR,10bR)-8,9-Dimethoxy-1,2,3,4,4a,10b-hexahydrophenanthridin-6-yl)-benzoylchloride hydrochloride is used.

12. N′-{1[3-((4aR,10bR)-8,9-Dimethoxy-1,2,3,4,a,10b-hexahydrophenanthridin-6-yl)-phenyl]-methanoyl}-N,N-diethylquanidine

M.p. 89-91° C.
MS: calc.: C₂₇H₃₄N₄O₃(462,6) fnd.: [M+1] 463,1
   13. 3-((4aR,10bR)-8,9-Dimethoxy-1,2,3,4,4a,10b-hexahydrophenanthridin-6-yl)-N-(1-imino-1-morpholin-4yl-methyl)-benzamide
M.p. 187-188° C.
MS: calc.: C₂₇H₃₂N₄O₄(476,58) fnd.: [M+1] 477,1
   Starting Materials
   A1. 4-[(4aR,10bR)8,9-Dimethoxy-1,2,3,4,4a,10b-hexahydrophenanthridin-6-yl]-benzoyl-chloride hydrochloride

50 g 4-[(4aR,10bR)-8,9-Dimethoxy-1,2,3,4,4a,10b-hexahydrophenanthridin-6-yl]-benzoic acid are suspended in 500 ml acetonitril and 500 ml methylene chloride at RT. 20 ml oxalyl chloride are added drop-wise and the mixture is stirred for 1 h. The solvents are removed under reduced pressure and the crude product is used without further purification.

A2. 4[(4aR,10bR)-8,9-Dimethoxy-1,2,3,4,4a,10b-hexahydrophenanthridin-6-yl)]-benzoic acid hydrochloride

17 g 4[(4aR,10bR)-8,9-Dimethoxy-1,2,3,4,4a,10b-hexahydro-phenanthridin-6-yl]-benzoic acid methyl ester are dissolved in 100 ml water and 50 ml conc. hydrochloric acid and stirred at 80° C. for 3 h. The solvent is removed under reduced pressure and the residue is crystallized from methyl ethyl ketone and methanol. After filtering and drying 12.8 g of the title compound are obtained of melting point 228° C. (decomp.).

A3. 3[(4aR,10bR)-8,9-Dimethoxy-1,2,3,4,4a,10b-hexahydrophenanthridin-6yl]-benzoylchloride hydrochloride

Prepared as described for starting compound A1.

A4. 3[(4aR,10bR)-8,9-Dimethoxy-1,2,3,4,4a.10b-hexahydrophenanthridin-6-yl-benzoic acid hydrochloride

Prepared as described for starting compound A2.
MS: calc.: C₂₂H₂₄Cl N O₄[365.43+(HCl)36.46] fnd.: [M+1] 366.2
   A5. 4-[(4aR,10bR)-8,9-Dimethoxy-1,2,3,4,4a.10b-hexahydro-phenanthridin-6-yl]-benzoic acid methyl ester

42.7 g N-[(1R,2R)-2-(3,4-Dimethoxy-phenyl)-cyclohexyl]-terephthalamic acid methyl ester and 25 ml phosphorus oxychloride are dissolved in 500 ml acetonitril and stirred overnight at 80° C. The solvent is evaporated under reduced pressure, the residue is dissolved in ethyl acetate and extracted with sodium bicarbonate solution. The organic layer is dried over sodium sulfate and concentrated. The crude product is purified by chromatography on silica gel using a mixture of petroleum ether/ethyl acetate/triethylamin in the ratio 6/3/1 to furnish 37.7 g of the title compound with a optical rotation of [α]_{D}²⁰=−82 (c=0.2, Ethanol).

A6. N-[(1R,2R)-2-(3,4-Dimethoxy-phenyl)-cyclohexyl]-terephthalamic acid methyl ester

27.2 g 1,2-Dimethoxy-4-[1R-(2R-aminocyclohexyl)]benzene are dissolved in 300 ml methylene chloride and 50 ml triethylamine. A solution of 27.2 g 4-chlorocarbonyl-benzoic acid methyl ester in 300 ml methylene chloride is added dropwise at room temperature and the mixture is stirred overnight. The solution is extracted with water, 1M hydrochloric acid, sodium bicarbonate solution and water. The organic layer is dried over sodium sulfate and the solvent is evaporated to yield 43.4 g of the title compound with melting point 154-156° C.

A7. 3((4aR,10bR)-8,9-Dimethoxy-1,2,3,4,4a.10b-hexahydro-phenanthridin-6-yl)-benzoic acid methyl ester

Prepared as described for starting compound A5. M.p. 110-111° C.

A8. N-[(1R,2R)-2-(3,4-Dimethoxy-phenyl)-cyclohexyl]-isophthalamic acid methyl ester

Prepared as described for starting compound A6. M.p. 108-109° C.

A9. 1,2-Dimethoxy-4-[1R-(2R-aminocyclohexyl)]benzene

12.0 g of a racemic mixture of 1,2-dimethoxy-4-[1R-(2R-aminocyclohexyl)]benzene and 1,2-dimethoxy-4-[1S(2S-aminocyclohexyl)]benzene and 6.2 g of (−)-mandelic acid are dissolved in 420 ml of dioxane and 60 ml of tetrahydrofuran and the solution is stirred overnight at RT. The solid is filtered off with suction, dried, treated with 100 ml of saturated sodium hydrogencarbonate solution and extracted with ethyl acetate. The organic phase is dried using sodium sulfate and concentrated under reduced pressure. 4.8 g of the title compound are obtained of m.p.: 80-81.5° C.
Specific rotation: [α]_{D}²⁰=−58.5° C. (c=1, ethanol).
   A10. 1.2-Dimethoxy-4-[1R-(2R-aminocyclohexyl)]benzene and 1,2-Dimethoxy-4-[1S-(2S-amino-cyclohexyl)]benzene

125 g of a racemic mixture of 1,2dimethoxy-4-[1R-(2R-nitrocyclohexyl)]benzene and 1,2-dimethoxy-4-[1S-(2S-nitrocyclohexyl)]benzene and 120 g of zinc powder or granules are suspended in 1300 ml of ethanol. 220 ml of acetic acid are added dropwise at boiling heat. The precipitate is filtered off with suction and washed with ethanol, and the filtrate is concentrated under reduced pressure. The residue is taken up in hydrochloric acid and extracted with toluene. The aqueous phase is rendered alkaline using 50% strength sodium hydroxide solution, the precipitate is filtered off with suction and the filtrate is extracted with toluene. The organic phase is dried using sodium sulfate and concentrated. 98 g of the title compounds are obtained as a crystallizing oil.

Alternatively:

8.5 g of a racemic mixture of 1,2-dimethoxy-4-[1R-(2R-nitrocyclohexyl)]benzene and 1,2-dimethoxy-4-[1S-(2S-nitrocyclohexyl)]benzene are dissolved in 400 ml of methanol and treated at RT with 7 ml of hydrazine hydrate and 2.5 g of Raney nickel in portions in the course of 8 h. After stirring overnight at RT, the reaction mixture is filtered, the filtrate is concentrated and the residue is chromatographed on silica gel using a mixture of toluene/ethyl acetate/triethylamine=4/2/0.5. The title compounds are obtained as an oil.

A11. 1.2-Dimethoxy-4-[1R-(2R-nitrocyclohexyl)]benzene and 1,2-Dimethoxy-4-[1S-(2S-nitrocyclohexyl)]benzene

8.4 g of a racemic mixture of 1,2-dimethoxy-4-[1R-(2R-nitrocyclohex-4-enyl)]benzene and 1,2-dimethoxy-4-[1R-(2R-nitrocyclohex-4-enyl)]benzene are dissolved in 450 ml of methanol, treated with 2 ml of conc. hydrochloric acid and hydrogenated after addition of 500 mg of 10% strength Pd/C. The reaction mixture is filtered and the filtrate is concentrated. M.p.: 84-86.5° C.

A12. 1.2-Dimethoxy-4-[1R-(2R-nitrocyclohex-4-enyl)]benzene and 1,2-Dimethoxy-4-[1S-(2S-nitrocyclohex-4-enyl)]benzene

10.0 g of a racemic mixture of 1,2-dimethoxy-4-[1S-(2R-nitrocyclohex-4-enyl)]benzene and 1,2-dimethoxy-4-[1R-(2S-nitrocyclohex-4-enyl)]benzene and 20.0 g of potassium hydroxide are dissolved in 150 ml of ethanol and 35 ml of dimethylformamide. A solution of 17.5 ml of conc. sulfuric acid in 60 ml of ethanol is then added dropwise such that the internal temperature does not exceed 4° C. After stirring for 1 h, the mixture is added to 1 l of ice water, the precipitate is filtered off with suction, washed with water and dried, and the crude product is recrystallized from ethanol. 8.6 g of the title compound of m.p. 82.5-84° C. are obtained.

A13. 1,2-Dimethoxy-4-[1S-(2R-nitrocyclohex-4-enyl)]benzene and 1,2-Dimethoxy-4-[1R-(2S-nitrocyclohex-4-enyl)]benzene

50.0 g of 3,4-dimethoxy-ω-nitrostyrene and 1.0 g (9.1 mmol) of hydroquinone are suspended in 200 ml of abs. toluene and treated at −70° C. with 55.0 g (1.02 mol) of liquid 1,3butadiene. The mixture is stirred at 160° C. for 6 days in an autoclave and then cooled. Some of the solvent is removed on a rotary evaporator, and the resulting precipitate is filtered off with suction and recrystallized in ethanol. M.p.: 113.5-115.5° C.

A14. 3.4Dimethoxy-ω-nitrostyrene

207.0 9 of 3,4-dimethoxybenzaldehyde, 100.0 g of ammonium acetate and 125 ml of nitromethane are heated to boiling for 3-4 h. in 1.0 l of glacial acetic acid. After cooling in an ice bath, the precipitate is filtered off with suction, rinsed with glacial acetic acid and petroleum ether and dried. M.p.: 140-141° C. Yield: 179.0 g.

### Commercial Utility

The compounds according to the invention have useful pharmacological properties which make them industrially utilizable. As selective cyclic nucleotide phosphodiesterase (PDE) inhibitors (specifically of type 4), they are suitable on the one hand as bronchial therapeutics (for the treatment of airway obstructions on account of their dilating action but also on account of their respiratory rate- or respiratory drive-increasing action) and for the removal of erectile dysfunction on account of their vascular dilating action, but on the other hand especially for the treatment of disorders, in particular of an inflammatory nature, e.g. of the airways (asthma prophylaxis), of the skin, of the intestine, of the eyes, of the CNS and of the joints, which are mediated by mediators such as histamine, PAF (platelet-activating factor), arachidonic acid derivatives such as leukotrienes and prostaglandins, cytokines, interleukins, chemokines, alpha-, beta- and gamma-interferon, tumor necrosis factor (TNF) or oxygen free radicals and proteases. In this context, the compounds according to the invention are distinguished by a low toxicity, a good enteral absorption (high bioavailability), a large therapeutic breadth and the absence of significant side effects.

On account of their PDE-inhibiting properties, the compounds according to the invention can be employed in human and veterinary medicine as therapeutics, where they can be used, for example, for the treatment and prophylaxis of the following illnesses: acute and chronic (in particular inflammatory and allergen-induced) airway disorders of varying origin (bronchitis, allergic bronchitis, bronchial asthma, emphysema, COPD); dermatoses (especially of proliferative, inflammatory and allergic type) such as psoriasis (vulgaris), toxic and allergic contact eczema, atopic eczema, seborrhoeic eczema, Lichen simplex, sunburn, pruritus in the anogenital area, alopecia areata, hypertrophic scars, discoid lupus erythematosus, follicular and widespread pyodermias, endogenous and exogenous acne, acne rosacea and other proliferative, inflammatory and allergic skin disorders; disorders which are based on an excessive release of TNF and leukotrienes, for example disorders of the arthritis type (rheumatoid arthritis, rheumatoid spondylitis, osteoarthritis and other arthritic conditions), disorders of the immune system (AIDS, multiple sclerosis), graft versus host reaction, allograft rejections, types of shock (septic shock, endotoxin shock, gram-negative sepsis, toxic shock syndrome and ARDS (adult respiratory distress syndrome)) and also generalized inflammations in the gastrointestinal region (Crohn's disease and ulcerative colitis); disorders which are based on allergic and/or chronic, immunological false reactions in the region of the upper airways (pharynx, nose) and the adjacent regions (paranasal sinuses, eyes), such as allergic rhinitis/sinusibs, chronic rhinitis/sinusitis, allergic conjunctivitis and also nasal polyps; but also disorders of the heart which can be treated by PDE inhibitors, such as cardiac insufficiency, or disorders which can be treated on account of the tissue-relaxant action of the PDE inhibitors, such as, for example, erectile dysfunction or colics of the kidneys and of the ureters in connection with kidney stones. In addition, the compounds of the invention are useful in the treatment of diabetes insipidus and conditions associated with cerebral metabolic inhibition, such as cerebral senility, senile dementia (Alzheimer's disease), memory impairment associated with Parkinson's disease or multiinfarct dementia; and also illnesses of the central nervous system, such as depressions or arteriosclerotic dementia.

The invention further relates to a method for the treatment of mammals, including humans, which are suffering from one of the above mentioned illnesses. The method is characterized in that a therapeutically active and pharmacologically effective and tolerable amount of one or more of the compounds according to the invention is administered to the ill mammal.

The invention further relates to the compounds according to the invention for use in the treatment and/or prophylaxis of illnesses, especially the illnesses mentioned.

The invention also relates to the use of the compounds according to the invention for the production of pharmaceutical compositions which are employed for the treatment and/or prophylaxis of the illnesses mentioned.

The invention furthermore relates to pharmaceutical compositions for the treatment and/or prophylaxis of the illnesses mentioned, which contain one or more of the compounds according to the invention.

Additionally, the invention relates to an article of manufacture, which comprises packaging material and a pharmaceutical agent contained within said packaging material, wherein the pharmaceutical agent is therapeutically effective for antagonizing the effects of the cyclic nucleotide phosphodiesterase of type 4 (PDE4), ameliorating the symptoms of an PDE4-mediated disorder, and wherein the packaging material comprises a label or package insert which indicates that the pharmaceutical agent is useful for preventing or treating PDE4-mediated disorders, and wherein said pharmaceutical agent comprises one or more compounds of formula 1 according to the invention. The packaging material, label and package insert otherwise parallel or resemble what is generally regarded as standard packaging material, labels and package inserts for pharmaceuticals having related utilities.

The pharmaceutical compositions are prepared by processes which are known per se and familiar to the person skilled in the art. As pharmaceutical compositions, the compounds according to the invention (=active compounds) are either employed as such, or preferably in combination with suitable pharmaceutical auxiliaries and/or excipients, e.g. in the form of tablets, coated tablets, capsules, caplets, suppositories, patches (e.g. as TTS), emulsions, suspensions, gels or solutions, the active compound content advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries or excipients which are suitable for the desired pharmaceutical formulations on account of his/her expert knowledge. In addition to solvents; gel formers, ointment bases and other active compound excipients, for example antioxidants, dispersants, emulsifiers, preservatives, solubilizers, colorants, complexing agents or permeation promoters, can be used.

The administration of the pharmaceutical compositions according to the invention may be performed in any of the generally accepted modes of administration available in the art Illustrative examples of suitable modes of administration include intravenous, oral, nasal, parenteral, topical, transdermal and rectal delivery. Oral delivery is preferred.

For the treatment of disorders of the respiratory tract, the compounds according to the invention are preferably also administered by inhalation in the form of an aerosol; the aerosol particles of solid, liquid or mixed composition preferably having a diameter of 0.5 to 10 μm, advantageously of 2 to 6 μm.

Aerosol generation can be carried out, for example, by pressure-driven jet atomizers or ultrasonic atomizers, but advantageously by propellant-driven metered aerosols or propellant-free administration of micronized active compounds from inhalation capsules.

Depending on the inhaler system used, in addition to the active compounds the administration forms additionally contain the required excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of apparatuses are available with which aerosols of optimum particle size can be generated and administered, using an inhalation technique which is as right as possible for the patient In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhaler described in European Patent Application EP 0 505 321), using which an optimal administration of active compound can be achieved.

For the treatment of dermatoses, the compounds according to the invention are in particular administered in the form of those pharmaceutical compositions which are suitable for topical application. For the production of the pharmaceutical compositions, the compounds according to the invention (=active compounds) are preferably mixed with suitable pharmaceutical auxiliaries and further processed to give suitable pharmaceutical formulations. Suitable pharmaceutical formulations are, for example, powders, emulsions, suspensions, sprays, oils, ointments, fatty ointments, creams, pastes, gels or solutions.

The pharmaceutical compositions according to the invention are prepared by processes known per se. The dosage of the active compounds is carried out in the order of magnitude customary for PDE inhibitors. Topical application forms (such as ointments) for the treatment of dermatoses thus contain the active compounds in a concentration of, for example, 0.1-99%. The dose for administration by inhalation is customarly between 0.1 and 3 mg per day. The customary dose in the case of systemic therapy (p.o. or i.v.) is between 0.03 and 3 mg/kg per day.

Biological Investigations

The second messenger cyclic AMP (cAMP) is well-known for inhibiting inflammatory and immunocompetent cells. The PDE4 isoenzyme is broadly expressed in cells involved in the initiation and propagation of inflammatory diseases (H Tenor and C Schudt, in “Phosphodiesterase Inhibitors”, 21-40, “The Handbook of Immunopharmacology”, Academic Press, 1996), and its inhibition leads to an increase of the intracellular cAMP concentration and thus to the inhibition of cellular activation (J E Souness et al., Immunopharmacology 47: 127-162, 2000).

The antiinflammatory potential of PDE4 inhibitors in vivo in various animal models has been described (MM Teixeira, TiPS 18: 164-170, 1997). For the investigation of PDE4 inhibition on the cellular level (in vitro), a large variety of proinflammatory responses can be measured. Examples are the superoxide production of neutrophilic (C Schudt et al., Arch Pharmacol 344: 682-690, 1991) or eosinophilic (A Hatzelmann et al., Brit J Pharmacol 114: 821-831, 1995) granulocytes, which can be measured as luminol-enhanced chemiluminescence, or the synthesis of tumor necrosis factor-α in monocytes, macrophages or dendritic cells (Gantner et al., Brit J Pharmacol 121: 221-231, 1997, and Pulmonary Pharmacol Therap 12: 377-386, 1999). In addition, the immunomodulatory potential of PDE4 inhibitors is evident from the inhibition of T-cell responses like cytokine synthesis or proliferation (D M Essayan, Biochem Pharmacol 57: 965-973, 1999). Substances which inhibit the secretion of the aforementioned proinflammatory mediators are those which inhibit PDE4. PDE4 inhibition by the compounds according to the invention is thus a central indicator for the suppression of inflammatory processes.

Method for Measuring Inhibition of PDE4 Activity

PDE4 activity was determined as described by Thompson et al. (Adv Cycl Nucl Res 10: 69-92, 1979) with some modifications (Bauer and Schwabe, Naunyn-Schmiedeberg's Arch Pharmacol 311: 193-198, 1980). At a final assay volume of 200 μl (96 well microtiter plates) the assay mixture contained 20 mM Tris (pH 7.4), 5 mM MgCl₂, 0.5 μM cAMP, [³H]cAMP (about 30,000 cpm/assay), the test compound and an aliquot of cytosol from human neutrophils which mainly contains PDE4 activity as described by Schudt et al. (Naunyn-Schmiedeberg's Arch Pharmacol 344: 682-690, 1991); the PDE3-specific inhibitor Motapizone (1 μM) was included to suppress PDE3 activity originating from contaminating platelets. Serial dilutions of the compounds were prepared in DMSO and further diluted 1:100 (vv) in the assays to obtain the desired final concentrations of the inhibitors at a DMSO concentration of 1% (v/v) which by itself only slightly affected PDE4 activity.

After preincubation for 5 min at 37° C., the reaction was started by the addition of substrate (cAMP) and the assays were incubated for further 15 min at 37° C. 50 μl of 0.2 N HCl was added to stop the reaction and the assays were left on ice for about 10 min. Following incubation with 25 μg 5′-nucleotidase (Crotalus atrox snake venom) for 10 min at 37° C., the assays were loaded on QAE Sephadex A-25 (1 ml bed volume). The columns were eluted with 2 ml of 30 mM ammonium formiate (pH 6.0) and the eluate was counted for radioactivity. Results were corrected for blank values (measured in the presence of denatured protein) which were below 5% of total radioactivity. The amount of cyclic nucleotides hydrolyzed did not exceed 30% of the original substrate concentration. The IC₅₀-values for the compounds according to the invention for the inhibition of the PDE4 activity were determined from the concentration-inhibition curves by nonlinear-regression.

For the following compounds inhibitory values [measured as -logIC₅₀ (mol/l)] higher than 8 were determined. The numbers of the compounds correspond to the numbers of the examples. Compounds 1-6 and 8-13.

1. A compound of formula 1, 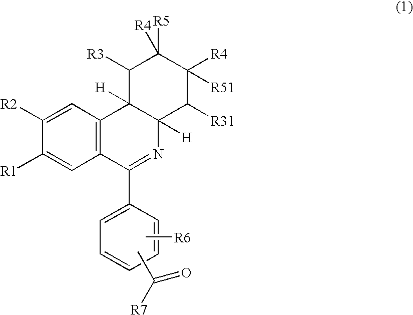
in which
R1 is hydroxyl, 1-4C-alkoxy, 3-7C-cycloalkoxy, 3-7C-cycloalkylmethoxy or completely or predominantly fluorine-substituted 1-4C-alkoxy, and
R2 is hydroxyl, 1-4C-alkoxy, 3-7C-cycloalkoxy, 3-7C-cycloalkylmethoxy or completely or predominantly fluorine-substituted 1-4C-alkoxy,
R1 and R2 together are a 1-2C-alkylenedioxy group,
R3 is hydrogen or 1-4C-alkyl,
R31 is hydrogen or 1-4C-alkyl,
or in which
R3 and R31 together are a 1-4C-alkylene group,
R4 is hydrogen or 1-4C-alkyl,
R5 is hydrogen,
R51 is hydrogen,
or in which
R5 and R51 together represent an additional bond,
R6 is hydrogen, halogen, nitro, 1-4C-alkyl, trifluoromethyl or 1-4C-alkoxy,
R7 is a radical of formulae (a), (b), (c) or (d) 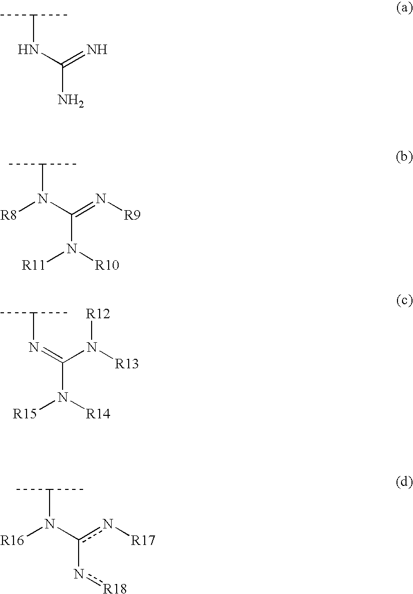
in which
if R7 is a radical of the formula (b),
either
R8, R9, R10 and R11 independently of one another are hydrogen, 1-7C-alkyl, 3-7C-cycloalkyl, 3-7C-cycloalkylmethyl, cyano, hydroxy-2-4C-alkyl, 1-4C-alkoxy-2-4C-alkyl or R28,
or
R8 is hydrogen, 1-7C-alkyl, 3-7C-cycloalkyl, 3-7C-cycloalkylmethyl, hydroxy-2-4C-alkyl, 1-4C-alkoxy-2-4C-alkyl or R28,
R9 is is hydrogen, 1-7C-alkyl, 3-7C-cycloalkyl, 3-7C-cycloalkylmethyl, hydroxy-2-4C-alkyl, 1-4C-alkoxy-2-4C-alkyl or R28, and
R10 and R11, together and including the nitrogen atom to which both are bonded, are a pyrrolidin-1-yl, piperidin-1-yl, azepan-1-yl, azocan-1-yl, azonan-1-yl, azecan-1-yl, morpholin-4-yl, tetrahydroisoquinolin-2-yl, tetrahydro-6,7-dimethoxyisoquinolin-2-yl, 3,5-dimethyl-pyrazol-1-yl, pyrazol-1-yl, 2,6-dimethyl-morpholin-4-yl, 2,6-dimethyl-piperidin-1-yl, 4-benzyl-piperidin-1-yl, thiomorpholin-4-yl or 1H-1,2,4-triazol-1-yl radical, or a piperazin-1-yl radical substituted in 4-position by R19,
or
R8 is hydrogen, 1-7C-alkyl, 3-7C-cycloalkyl, 3-7C-cycloalkylmethyl, hydroxy-2-4C-alkyl, 1-4C-alkoxy-2-4C-alkyl or R28,
R9 is hydrogen, 1-7C-alkyl, 3-7C-cycloalkyl, 3-7C-cycloalkylmethyl, hydroxy-2-4C-alkyl, 1-4C-alkoxy-2-4C-alkyl or R28,
R10 is hydrogen, 1-7C-alkyl, 3-7C-cycloalkyl, 3-7C-cycloalkylmethyl, hydroxy-2-4C-alkyl, 1-4C-alkoxy-2-4C-alkyl or R28, and
R11 is Aryl1, naphthyl, phenyl, phenyl substituted by R20 and/or R21, phenyl-1-4C-alkyl or phenyl-1-4C-alkyl substituted by R22 and R23,
in which
if R7 is a radical of the formula (c),
either
R12, R13, R14 and R15 independently of one another are hydrogen, 1-7C-alkyl, 3-7C-cycloalkyl, 3-7C-cycloalkylmethyl, hydroxy-2-4C-alkyl, 1-4C-alkoxy-2-4C-alkyl or R28,
or
R12 and R13 independently of one another are hydrogen, 1-7C-alkyl, 3-7C-cycloalkyl, 3-7C-cycloalkylmethyl, hydroxy-2-4C-alkyl, 1-4C-alkoxy-2-4C-alkyl or R28, and R14 and R15, together and including the nitrogen atom to which both are bonded, are a pyrrolidin-1-yl, piperidin-1-yl, azepan-1-yl, azocan-1-yl, azonan-1-yl, azecan-1-yl, morpholin-4-yl, tetrahydroisoquinolin-2-yl, tetrahydro-6,7-dimethoxyisoquinolin-2-yl, 3,5-dimethyl-pyrazol-1-yl, pyrazol-1-yl, 2,6-dimethyl-morpholin-4-yl, 2,6-dimethyl-piperidin-1-yl, 4-benzyl-piperidin-1-yl, thiomorpholin-4-yl or 1H-1,2,4-triazol-1-yl radical, or a piperazin-1-yl radical substituted in 4-position by R19,
or
R12 and R13, together and including the nitrogen atom to which both are bonded, are a pyrrolidin-1-yl, piperidin-1-yl, azepan-1-yl, morpholino-4-yl, 4-(1-4C-alkyl-)-piperazin-1-yl, 2,6-dimethyl-morpholin-4-yl, 2,6-dimethyl-piperidin-1-yl, 4-benzyl-piperidin-1-yl or thiomorpholin-4-yl radical, and
R14 and R15, together and including the nitrogen atom to which both are bonded, are a pyrrolidin-1-yl, piperidin-1-yl, azepan-1-yl, morpholino-4-yl, 4-(1-4C-alkyl-)-piperazin-1-yl, 2,6-dimethyl-morpholin-4-yl, 2,6-dimethyl-piperidin-1-yl, 4-benzyl-piperidin-1-yl or thiomorpholin-4-yl radical,
or
R12 and R15 independently of one another are hydrogen or 1-4C-alkyl, and
R13 and R14, together and with inclusion of the N—C(═)—N structure to which they are bonded, are a hexahydropyrimidin-2-ylidene or imidazolidin-2-ylidene radical,
in which
if R7 is a radical of the formula (d),
R16 is hydrogen, 1-7C-alkyl, 3-7C-cycloalkyl, 3-7C-cycloalkylmethyl, hydroxy-2-4C-alkyl, 1-4C-alkoxy-2-4C-alkyl or R28, and
R17 and R18, together and with inclusion of the N—C(−)—N structure to which they are bonded are Aryl2,
Aryl1 is 4-methylthiazol-2-yl, benzimidazol-2-yl, 5-nitrobenzimidazol-2-yl, 5-chlorobenzimidazol-2-yl, 5-methylbenzimidazol-2-yl, 4-methylquinazolin-2-yl, benzothiazol-2-yl, benzoxazol-2-yl or pyrimidin-2-yl,
Aryl2 is 1-methyl-4-oxo-4,5-dihydro-1H-imidazol-2-yl, imidazol-2-yl, 4,5-dicyano-imidazol-2-yl, 4-methyl-imidazol-2-yl, 4-ethyl-benzimidazol-2-yl, 4-acetyl-imidazol-2-yl, 1H-[1,2,4]triazol-3-yl, benzimidazol-2-yl, 1-methyl-benzimidazol-2-yl, 1-ethyl-benzimidazol-2-yl, 5,6-dimethyl-benzimidazol-2-yl, purin-8-yl, 6-amino-7-methyl-7H-purine-8-yl, 1,6-dimethylimidazo[4,5-b]pyridin-2-yl, 1,5,6-trimethylimidazo[4,5-b]pyridin-2-yl, 1,3-dimethyl-3,7-dihydro-1H-purine-2,6-dione-8-yl, 7-ethyl-3-methyl-3,7-dihydro-purine-2,6-dione-8-yl, 1,3,7-trimethyl-3,7-dihydro-purine-2,6-dione-8-yl, thiadiazolyl, 1,4-dihydrotetrazol-5-yl, 2H-[1,2,4]triazol-3-yl, 1,3-dihydrobenzimidazol-5-yl, 1H-tetrazol-5-yl, pyrimidin-2-yl or 4,6-dimethyl-pyrimidin-2-yl,
R19 is 1-4C-alkyl, formyl, 3-7C-cycloalkyl, 3-7C-cycloalkylmethyl, 1-4C-alkoxycarbonyl-1-4C-alkyl, 1-4C-alkylcarbonyl, hydroxy-2-4C-alkyl, 1-4C-alkoxy-2-4C-alkyl, hydroxy-2-4C-alkoxy-2-4C-alkyl, 1-4C-alkoxy-2-4C-alkoxy-2-4C-alkyl, phenyl, phenyl substituted by R24 and/or R25, [benzo(1,3)dioxol]-5-ylmethyl, phenyl-1-4C-alkyl or phenyl-1-4C-alkyl substituted in the phenyl moiety by R26 and/or R27,
R20 is halogen, nitro, carboxyl, 1-4C-alkyl, trifluoromethyl or 1-4C-alkoxy,
R21 is halogen, 1-4C-alkyl or 1-4C-alkoxy,
R22 is halogen, nitro, carboxyl, 1-4C-alkyl, trifluoromethyl or 1-4C-alkoxy,
R23 is halogen, 1-4C-alkyl or 1-4C-alkoxy,
R24 is halogen, nitro, carboxyl, 1-4C-alkyl, 1-4C-alkylcarbonyl, trifluoromethyl or 1-4C-alkoxy,
R25 is halogen, 1-4C-alkyl or 1-4C-alkoxy,
R26 is halogen, nitro, carboxyl, 1-4C-alkyl, trifluoromethyl or 1-4C-alkoxy,
R27 is halogen, 1-4C-alkyl or 1-4C-alkoxy,
R28 is R29(R30)N-2-4C-alkyl wherein
R29 and R30, together and including the nitrogen atom to which both are bonded, are a pyrrolidin-1-yl, piperidin-1-yl, piperazin-1-yl, 4-(1-4C-alkyl-)piperazin-1-yl, azepan-1yl, azocan-1-yl, azonan-1-yl, azecan-1-yl, tetrahydroisoquinolin-2-yl, tetrahydro-6,7-dimethoxyisoquinolin-2-yl, 3,5-dimethyl-pyrazol-1-yl, pyrazol-1-yl, morpholin-4-yl, 2,6-dimethyl-morpholin-4-yl, 2,6-dimethyl-piperidin-1-yl, 4-benzyl-piperidin-1-yl, thiomorpholin-4-yl or 1H-1,2,4-triazol-1-yl radical, or a hydrate, solvate, salt, hydrate of a salt or solvate of a salt of this compound, or an N-oxide, enantiomer, E/Z isomer or tautomer of this compound, or a salt thereof.

2. A compound of formula 1 as claimed in claim 1, in which
R1 is hydroxyl, 1-4C-alkoxy, 3-7C-cycloalkoxy, 3-7C-cycloalkylmethoxy or completely or predominantly fluorine-substituted 1-4C-alkoxy, and
R2 is hydroxyl, 1-4C-alkoxy, 3-7C-cycloalkoxy, 3-7C-cycloalkylmethoxy or completely or predominantly fluorine-substituted 1-4C-alkoxy,
R1 and R2 together are a 1-2C-alkylenedioxy group,
R3 is hydrogen or 1-4C-alkyl,
R31 is hydrogen or 1-4C-alkyl, or in which
R3 and R31 together are a 1-4C-alkylene group,
R4 is hydrogen or 1-4C-alkyl,
R5 is hydrogen,
R51 is hydrogen,
or in which
R5 and R51 together represent an additional bond,
R6 is hydrogen, halogen, nitro, 1-4C-alkyl, trifluoromethyl or 1-4C-alkoxy,
R7 is a radical of formulae (a), (b), (c) or (d) 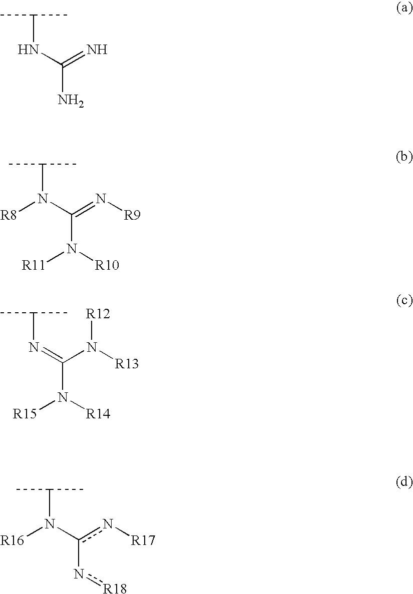
in which
if R7 is a radical of the formula (b),
either
R8, R9, R10 and R11 independently of one another are hydrogen, 1-7C-alkyl, 3-7C-cycloalkyl, 3-7C-cycloalkylmethyl or hydroxy-2-4C-alkyl,
or
R8 is hydrogen, 1-7C-alkyl, 3-7C-cycloalkyl, 3-7C-cycloalkylmethyl or hydroxy-2-4C-alkyl,
R9 is hydrogen, 1-7C-alkyl, 3-7C-cycloalkyl, 3-7C-cycloalkylmethyl or hydroxy-2-4C-alkyl, and
R10 and R11, together and including the nitrogen atom to which both are bonded, are a pyrrolidin-1-yl, piperidin-1-yl, azepan-1-yl, azocan-1-yl, azonan-1-yl, azecan-1-yl, morpholin-4-yl, tetrahydroisoquinolin-2-yl, 3,5-dimethyl-pyrazol-1-yl, pyrazol-1-yl, 2,6-dimethyl-morpholin-4-yl, 2,6-dimethyl-piperidin-1-yl, thiomorpholin-4-yl or 1H-1,2,4-triazol-1-yl radical, or a piperazin-1-yl radical substituted in 4-position by R19,
or
R8 is hydrogen, 1-7C-alkyl, 3-7C-cycloalkyl, 3-7C-cycloalkylmethyl or hydroxy-2-4C-alkyl,
R9 is hydrogen, 1-7C-alkyl, 3-7C-cycloalkyl, 3-7C-cycloalkylmethyl or hydroxy-2-4C-alkyl,
R10 is hydrogen, 1-7C-alkyl, 3-7C-cycloalkyl, 3-7C-cycloalkylmethyl or hydroxy-2-4C-alkyl, and
R11 is Aryl1, naphthyl, phenyl, phenyl substituted by
R20 and/or R21, phenyl-1-4C-alkyl or phenyl-1-4C-alkyl substituted by R22 and R23,
in which
if R7 is a radical of the formula (c),
either
R12, R13, R14 and R15 independently of one another are hydrogen, 1-7C-alkyl, 3-7C-cycloalkyl, 3-7C-cycloalkylmethyl or hydroxy-2-4C-alkyl,
or
R12 and R13 independently of one another are hydrogen, 1-7C-alkyl, 3-7C-cycloalkyl, 3-7C-cycloalkylmethyl or hydroxy-2-4C-alkyl, and
R14 and R15, together and including the nitrogen atom to which both are bonded, are a pyrrolidin-1-yl, piperidin-1-yl, azepan-1-yl, azocan-1-yl, azonan-1-yl, azecan-1-yl, morpholin-4-yl, tetrahydroisoquinolin-2-yl, 3,5-dimethyl-pyrazol-1-yl, pyrazol-1-yl, 2,6-dimethyl-morpholin-4-yl, 2,6-dimethyl-piperidin-1-yl, thiomorpholin-4-yl or 1H-1,2,4-triazol-1-yl radical, or a piperazin-1-yl radical substituted in 4-position by R19,
or
R12 and R13, together and including the nitrogen atom to which both are bonded, are a pyrrolidin-1-yl, piperidin-1-yl, azepan-1-yl, morpholino-4-yl, 4-(1-4C-alkyl-)-piperazin-1-yl, 2,6-dimethyl-morpholin-4-yl, 2,6-dimethyl-piperidin-1-yl or thiomorpholin-4-yl radical, and
R14 and R15, together and including the nitrogen atom to which both are bonded, are a pyrrolidin-1-yl, piperidin-1-yl, azepan-1-yl, morpholino-4-yl, 4-(1-4C-alkyl-)-piperazin-1-yl, 2,6-dimethyl-morpholin-4-yl, 2,6-dimethyl-piperidin-1-yl or thiomorpholin-4-yl radical,
or
R12 and R15 independently of one another are hydrogen or 1-4C-alkyl, and
R13 and R14, together and with inclusion of the N—C(═)—N structure to which they are bonded, are a hexahydropyrimidin-2-ylidene or imidazolidin-2-ylidene radical,
in which
if R7 is a radical of the formula (d),
R16 is hydrogen, 1-7C-alkyl, 3-7C-cycloalkyl, 3-7C-cycloalkylmethyl or hydroxy-2-4C-alkyl, and
R17 and R18, together and with inclusion of the N—C(−)—N structure to which they are bonded are Aryl2,
Aryl1 is 4-methylthiazol-2-yl, benzimidazol-2-yl, 5-nitrobenzimidazol-2-yl, 5-chlorobenzimidazol-2-yl, 5-methylbenzimidazol-2-yl, 4-methylquinazolin-2-yl, benzothiazol-2-yl, benzoxazol-2-yl or pyrimidin-2-yl,
Aryl2 is 1-methyl-4-oxo-4,5-dihydro-1H-imidazol-2-yl, imidazol-2-yl, 4,5-dicyano-imidazol-2-yl, 4-methyl-imidazol-2-yl, 4-ethyl-benzimidazol-2-yl, 4-acetyl-imidazol-2-yl, 1H-[1,2,4]triazol-3-yl, benzimidazol-2-yl, 1-methyl-benzimidazol-2-yl, 1-ethyl-benzimidazol-2-yl, 5,6-dimethyl-benzimidazol-2-yl, purin-8-yl, 6-amino-7-methyl-7H-purine-8-yl, 1,6-dimethylimidazo[4,5-b]pyridin-2-yl, 1,5,6-trimethylimidazo[4,5-b]pyridin-2-yl, 1,3-dimethyl-3,7-dihydro-1H-purine-2,6-dione-8-yl, 7-ethyl-3-methyl-3,7-dihydro-purine-2,6-dione-8-yl, 1,3,7-trimethyl-3,7-dihydro-purine-2,6-dione-8-yl, thiadiazolyl, 1,4-dihydrotetrazol-5-yl, 2H-(1,2,4]triazol-3-yl, 1,3-dihydrobenzimidazol-5-yl, 1H-tetrazol-5-yl, pyrimidin-2-yl or 4,6-dimethyl-pyrimidin-2-yl,
R19 is 1-4C-alkyl, formyl, 1-4C-alkylcarbonyl, 2-hydroxyethyl, phenyl, phenyl substituted by R24 and/or R25, phenyl-1-4C-alkyl or phenyl-1-4C-alkyl substituted in the phenyl moiety by R26 and/or R27,
R20 is halogen, nitro, carboxyl, 1-4C-alkyl, trifluoromethyl or 1-4C-alkoxy,
R21 is halogen, 1-4C-alkyl or 1-4C-alkoxy,
R22 is halogen, nitro, carboxyl, 1-4C-alkyl, trifluoromethyl or 1-4C-alkoxy,
R23 is halogen, 1-4C-alkyl or 1-4C-alkoxy,
R24 is halogen, nitro, carboxyl, 1-4C-alkyl, trifluoromethyl or 1-4C-alkoxy,
R25 is halogen, 1-4C-alkyl or 1-4C-alkoxy,
R26 is halogen, nitro, carboxyl, 1-4C-alkyl, trifluoromethyl or 1-4C-alkoxy,
R27 is halogen, 1-4C-alkyl or 1-4C-alkoxy, or a hydrate, solvate, salt, hydrate of a salt or solvate of a salt of this compound, or an N-oxide, enantiomer, E/Z isomer or tautomer of this compound, or a salt thereof.

3. A compound of formula 1 as claimed in claim 1, in which
R1 is 1-2C-alkoxy, 3-5C-cycloalkoxy, 3-5C-cycloalkylmethoxy or completely or predominantly fluorine-substituted 1-2C-alkoxy,
R2 is 1-2C-alkoxy, 3-5C-cycloalkoxy, 3-5C-cycloalkylmethoxy or completely or predominantly fluorine-substituted 1-2C-alkoxy,
R3 is hydrogen,
R31 is hydrogen,
R4 is hydrogen or 1-2C-alkyl,
R5 is hydrogen,
R51 is hydrogen,
or in which
R5 and R51 together represent an additional bond,
R6 is hydrogen, halogen, nitro, 1-4C-alkyl, trifluoromethyl or 1-4C-alkoxy,
R7 is a radical of formulae (a), (b), (c) or (d) 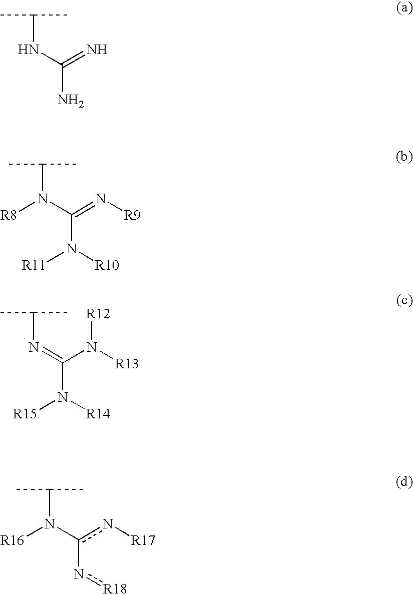
in which
if R7 is a radical of the formula (b),
either
R8 is hydrogen, and
R9, R10 and R11 independently of one another are hydrogen, 1-4C-alkyl, 3-7C-cycloalkyl or 3-7C-cycloalkylmethyl,
or
R8 is hydrogen,
R9 is hydrogen, 1-4C-alkyl, 3-7C-cycloalkyl or 3-7C-cycloalkylmethyl, and
R10 and R11, together and including the nitrogen atom to which both are bonded, are a pyrrolidin-1-yl, piperidin-1-yl, azepan-1-yl, azocan-1-yl, azonan-1-yl, azecan-1-yl, morpholin-4-yl, tetrahydroisoquinolin-2-yl, 3,5-dimethyl-pyrazol-1-yl, pyrazol-1-yl, 2,6-dimethyl-morpholin-4-yl or 2,6-dimethyl-piperidin-1-yl radical, or a piperazin-1-yl radical substituted in 4-position by R19,
or
R8 is hydrogen,
R9 is hydrogen, 1-7C-alkyl, 3-7C-cycloalkyl or 3-7C-cycloalkylmethyl,
R10 is hydrogen, 1-7C-alkyl, 3-7C-cycloalkyl or 3-7C-cycloalkylmethyl, and
R11 is Aryl1, naphthyl, phenyl, phenyl substituted by R20 and/or R21, phenyl-1-4C-alkyl or phenyl-1-4C-alkyl substituted by R22 and R23,
in which
if R7 is a radical of the formula (c),
either
R12, R13, R14 and R15 independently of one another are hydrogen, 1-4C-alkyl, 3-7C-cycloalkyl or 3-7C-cycloalkylmethyl,
or
R12 and R13 independently of one another are hydrogen, 1-4C-alkyl, 3-7C-cycloalkyl or 3-7C-cycloalkylmethyl, and
R14 and R15, together and including the nitrogen atom to which both are bonded, are a pyrrolidin-1-yl, piperidin-1-yl, azepan-1-yl, azocan-1-yl, azonan-1-yl, azecan-1-yl, morpholin-4-yl, tetrahydroisoquinolin-2-yl, 3,5-dimethyl-pyrazol-1-yl, pyrazol-1-yl, 2,6-dimethyl-morpholin-4-yl or 2,6-dimethyl-piperidin-1-yl radical, or a piperazin-1-yl radical substituted in 4-position by R19,
or
R12 and R13, together and including the nitrogen atom to which both are bonded, are a pyrrolidin-1-yl, piperidin-1-yl, azepan-1-yl, morpholino-4-yl, 4-(1-4C-alkyl-)-piperazin-1-yl, 2,6-dimethyl-morpholin-4-yl or 2,6-dimethyl-piperidin-1-yl radical, and
R14 and R15, together and including the nitrogen atom to which both are bonded, are a pyrrolidin-1-yl, piperidin-1-yl, azepan-1-yl, morpholino-4-yl, 4- (1-4C-alkyl-) -piperazin-1-yl, 2,6-dimethyl-morpholin-4-yl or 2,6-dimethyl-piperidin-1-yl radical, and
or
R12 and R15 independently of one another are hydrogen or 1-4C-alkyl, and
R13 and R14, together and including the N—C(═)—N structure to which they are bonded, are a hexahydropyrimidin-2-ylidene or imidazolidin-2-ylidene radical,
in which
if R7 is a radical of the formula (d),
R16 is hydrogen, and
R17 and R18, together and with inclusion of the N—C(−)—N structure to which they are bonded are Aryl2,
Aryl1 is 4-methylthiazol-2-yl, benzimidazol-2-yl, 5-nitrobenzimidazol-2-yl, 5-chlorobenzimidazol-2-yl, 5-methylbenzimidazol-2-yl, benzothiazol-2-yl or benzoxazol-2-yl,
Aryl2 is 1-methyl-4-oxo-4,5-dihydro-1H-imidazol-2-yl, imidazol-2-yl, 4,5-dicyano-imidazol-2-yl, 4-methyl-imidazol-2-yl, 4-ethyl-benzimidazol-2-yl, 4-acetyl-imidazol-2-yl, 1H-[1,2,4]triazol-3-yl, benzimidazol-2-yl, 1-methyl-benzimidazol-2-yl, 1-ethyl-benzimidazol-2-yl, 5,6-dimethyl-benzimidazol-2-yl, purin-8-yl, 6-amino-7-methyl-7H-purine-8-yl, 1,6-dimethylimidazo[4,5-b]pyridin-2-yl, 1,5,6-trimethylimidazo[4,5-b]pyridin-2-yl, 1,3-dimethyl-3,7-dihydro-1H-purine-2,6-dione-8-yl, 7-ethyl-3-methyl-3,7-dihydro-purine-2,6-dione-8-yl, 1,3,7-trimethyl-3,7-dihydro-purine-2,6-dione-8-yl or 1H-[1,2,4]triazol-3-yl,
R19 is 1-4C-alkyl, formyl, 1-4C-alkylcarbonyl, 2-hydroxyethyl, phenyl, phenyl substituted by R24 and/or R25, phenyl-1-4C-alkyl or phenyl-1-4C-alkyl substituted in the phenyl moiety by R26 and/or R27,
R20 is halogen, nitro, 1-4C-alkyl or 1-4C-alkoxy,
R21 is halogen, 1-4C-alkyl or 1-4C-alkoxy,
R22 is halogen, nitro, 1-4C-alkyl or 1-4C-alkoxy,
R23 is halogen, 1-4C-alkyl or 1-4C-alkoxy,
R24 is halogen, nitro, 1-4C-alkyl or 1-4C-alkoxy,
R25 is halogen, 1-4C-alkyl or 1-4C-alkoxy,
R26 is halogen, nitro, 1-4C-alkyl or 1-4C-alkoxy,
R27 is halogen, 1-4C-alkyl or 1-4C-alkoxy, or a hydrate, solvate, salt, hydrate of a salt or solvate of a salt of this compound, or an N-oxide, enantiomer, E/Z isomer or tautomer of this compound, or a salt thereof.

4. A compound of formula 1 as claimed in claim 1, in which
R1 is 1-2C-alkoxy,
R2 is 1-2C-alkoxy,
R3, R31, R4, R5 and R51 are hydrogen,
R6 is hydrogen,
R7 is a radical of formulae (a), (b), (c) or (d) 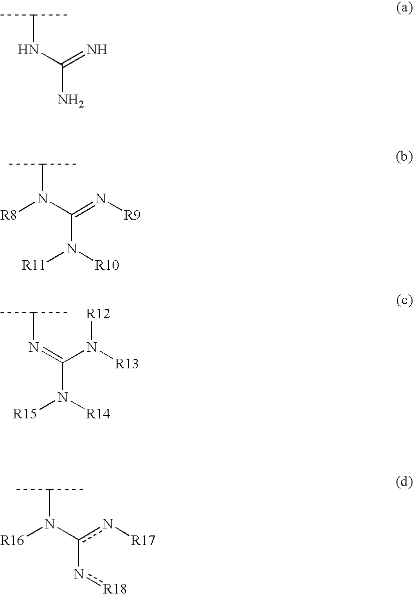
in which
if R7 is a radical of the formula (b),
either
R8 is hydrogen,
R9 is hydrogen,
R10 is hydrogen or 1-4C-alkyl,
R11 is hydrogen or 1-4C-alkyl, where at least one of the radicals R10 or R11 is not hydrogen,
or
R8 is hydrogen,
R9 is hydrogen,
R10 and R11, together and including the nitrogen atom to which both are bonded, are a pyrrolidin-1-yl, piperidin-1-yl, azepan-1-yl, azocan-1-yl, azonan-1-yl, morpholin-4-yl, tetrahydroisoquinolin-2-yl or 3,5-dimethyl-pyrazol-i-yl radical, or a piperazin-1-yl radical substituted by R19,
or
R8 is hydrogen,
R9 is hydrogen,
R10 is hydrogen or 1-4C-alkyl, and
R11 is Aryl1, naphthyl, phenyl or phenyl substituted by R20,
in which
if R7 is a radical of the formula (c),
either
R12 is hydrogen or 1-4C-alkyl,
R13 is hydrogen or 1-4C-alkyl,
R14 is hydrogen or 1-4C-alkyl, and
R15 is hydrogen or 1-4C-alkyl,
where at least one of the radicals R12, R13, R14 and R15 is not hydrogen,
or
R12 is hydrogen or 1-4C-alkyl,
R13 is hydrogen or 1-4C-alkyl, and
R14 and R15, together and including the nitrogen atom to which both are bonded, are a pyrrolidin-1-yl, piperidin-1-yl, azepan-1-yl, azocan-1-yl, azonan-1-yl, morpholin-4-yl, tetrahydroisoquinolin-2-yl or 3,5-dimethyl-pyrazol-1-yl radical, or a piperazin-1-yl radical substituted by R19,
in which
if R7 is a radical of the formula (d),
R16 is hydrogen, and
R17 and R18, together and with inclusion of the N—C(−)—N structure to which they are bonded are Aryl2,
Aryl1 is benzimidazol-2-yl, 5-nitrobenzimidazol-2-yl, 5-chlorobenzimidazol-2-yl or 5-methylbenzimidazol-2-yl,
Aryl2 is imidazol-2-yl, 4-methyl-imidazol-2-yl, 4-ethyl-benzimidazol-2-yl, 4-acetyl-imidazol-2-yl, 1H-[1,2,4]triazol-3-yl, benzimidazol-2-yl, 1-methyl-benzimidazol-2-yl, 1-ethyl-benzimidazol-2-yl, 5,6-dimethyl-benzimidazol-2-yl, purin-8-yl, 1,6-dimethylimidazo[4,5-b]pyridin-2-yl, 1,5,6-trimethyl-imidazo[4,5-b]pyridin-2-yl,
R19 is 1-4C-alkyl,
R20 is halogen, nitro, 1-4C-alkyl or 1-4C-alkoxy, or a hydrate, solvate, salt, hydrate of a salt or solvate of a salt of this compound, or an N-oxide, enantiomer, E/Z isomer or tautomer of this compound, or a salt thereof.

5. A compound of formula 1 as claimed in claim 1, in which
R1 is methoxy,
R2 is methoxy,
R3, R31, R4, R5 and R51 are hydrogen,
R6 is hydrogen,
R7 is N′-(N,N-diethyl)guanidinyl, 1H-imidazol-2-yl-amino, (morpholine)-4-carboxamidinyl, N′-(N,N-dimethyl)guanidinyl, 4-methylpiperazine-1-carboxamidinyl, 1H-[1,2,4]triazol-3-yl-amino, N′-[N-(1H-benzimidazol-2-yl)]guanidinyl, 2-(tetrahydroisoquinoline)-carboxamidinyl, pyrrolidin-1-carboxamidinyl, N′-(N-phenyl)guanidinyl or 3,5-dimethylpyrazol-1-carboxamidinyl, or a hydrate, solvate, salt, hydrate of a salt or solvate of a salt of this compound, or an N-oxide, enantiomer, E/Z isomer or tautomer of this compound, or a salt thereof.

6. A compound of formula 1 as claimed in claim 1 in which
R1 is methoxy,
R2 is methoxy,
R3, R31, R4, R5 and R51 are hydrogen,
R6 is hydrogen,
R7 is a radical selected from the group consisting of 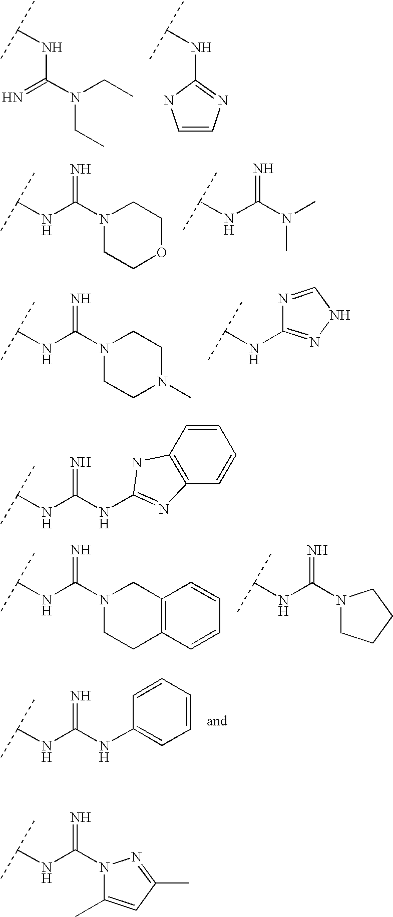
or a hydrate, solvate, salt, hydrate of a salt or solvate of a salt of this compound, or an N-oxide, enantiomer, E/Z isomer or tautomer of this compound, or a salt thereof.

7. A compound of formula 1 as claimed in claim 1, in which the hydrogen atoms in positions 4a and 10b are in the cis position relative to one another, or a hydrate, solvate, salt, hydrate of a salt or solvate of a salt of this compound, or an N-oxide, enantiomer, E/Z isomer or tautomer of this compound, or a salt thereof.

8. A compound of formula 1 as claimed in claim 1, which has with respect to the positions 4a and 10b the configuration shown in formula (1*): 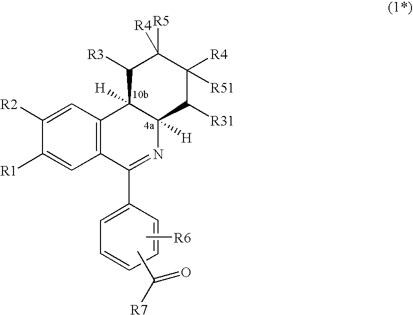
or a hydrate, solvate, salt, hydrate of a salt or solvate of a salt of this compound, or an N-oxide, enantiomer, E/Z isomer or tautomer of this compound, or a salt thereof.

9. (canceled)

10. A pharmaceutical composition comprising one or more compounds of formula 1 as claimed in claim 1, or a pharmaceutically acceptable hydrate, solvate, salt, hydrate of a salt or solvate of a salt of this compound,
or a pharmaceutically acceptable N-oxide, enantiomer, E/Z isomer or tautomer of this compound, or a salt thereof, together with a pharmaceutical auxiliary and/or excipient.

11. (canceled)

12. A method for treating an illness treatable by administration of a PDE4 inhibitor in a patient comprising administering to said patient in need thereof a therapeutically effective amount of a compound of formula 1 as claimed in claim 1, or a pharmaceutically acceptable hydrate, solvate, salt, hydrate of a salt or solvate of a salt of this compound,
or a pharmaceutically acceptable N-oxide, enantiomer, E/Z isomer or tautomer of this compound, or a salt thereof.

13. A method for treating an airway disorder in a patient comprising administering to said patient a therapeutically effective amount of a compound of formula 1 as claimed in claim 1, or a pharmaceutically acceptable hydrate, solvate, salt, hydrate of a salt or solvate of a salt of this compound,
or a pharmaceutically acceptable N-oxide, enantiomer, E/Z isomer or tautomer of this compound, or a salt thereof.

14. A method for treating dermatoses in a patient comprising administering to said patient a therapeutically effective amount of a compound of formula 1 according to claim 1, or a pharmaceutically acceptable hydrate, solvate, salt, hydrate of a salt or solvate of a salt of this compound,
or a pharmaceutically acceptable N-oxide, enantiomer, E/Z isomer or tautomer of this compound, or a salt thereof.
